(19) United States
(12) Patent Application Publication
(10) Pub. No.: US 2011/0112195 A1
(43) Pub. Date: May 12, 2011

(54) Potent and Selective Inhibition by Aurinticarboxylic Acid

(76) Inventors: Runtao He, Winnipeg (CA); Anton Andonov, Winnipeg (CA); Jingxin Cao, Winnipeg (CA); Mike Drebot, Winnipeg (CA); Xueguang Li, Winnipeg (CA)

(21) Appl. No.: 11/570,650

(22) PCT Filed: Jun. 15, 2005
(86) PCT No.: PCT/CA2005/000924

§ 371 Date: Dec. 1, 2008

Related U.S. Application Data
(60) Provisional application No. 60/579,247, filed on Jun. 15, 2004.
(60) Provisional application No. 60/608,862, filed on Sep. 13, 2004.

(51) Int. Cl. A61K 31/194; C40B 30/02

(52) U.S. Cl. 514/568; 506/8

(57) ABSTRACT

The severe acute respiratory syndrome virus (SARS) is a coronavirus that instigated regional epidemics in Canada and several Asian countries in 2003. The newly identified SARS coronavirus (SARS-CoV) can be transmitted among humans and cause severe or even fatal illnesses. As preventive vaccine development takes years to complete and adverse reactions have been reported to some veterinary coronaviral vaccines, anti-viral compounds must be relentlessly pursued. In this study, we analyzed the effect of aurintricarboxylic acid (ATA) on SARS-CoV replication in cell culture, and found that ATA could drastically inhibit SARS-CoV replication, with viral production being more than 1000 fold than that in the untreated control. ATA is also shown to be an effective anti-viral for several other viruses, including West Nile Virus and variola virus.

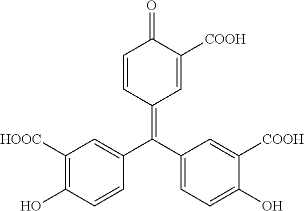

### PRIOR APPLICATION INFORMATION

This application claims the benefit of U.S. Provisional Application 60/579,247, filed Jun. 15, 2004 and U.S. Provisional Application 60/698,862, filed Sep. 13, 2004.

### BACKGROUND OF THE INVENTION

A new coronavirus that caused severe acute respiratory syndrome (SARS) was identified in early 2003, and subsequently named SARS coronavirus (SARS-CoV). The virus has a high tendency to spread among humans, and the mortality can be as high as 10-15% [1,2]. The complete understanding of pathogenesis of SARS remains tentative: a recent histological study using SARS-CoV infected patient lung samples found that diffuse alveolar damage may play an important role in the progression of the disease [3]. Even though there was a significant morbidity drop this year, the likelihood of the evolution of SARS-CoV in humans and animals may result in a re-emergence of the deadly virus.

Coronaviruses are enveloped viruses with single-stranded positive-sense RNA genomes typically of approximately 30 kb [4]. Most viruses in the coronavirus family cause diseases in animals, while a few, such as HCoV-229E, HCoV 0043, HCoV-NL-63 and SARS-CoV, are human pathogens [5,6,7]. Among all human coronaviruses, SARS-CoV is the only one that causes severe clinical consequences. Sequence comparisons of SARS-CoV genome sequences from different patient isolates revealed high homology; however, the sequence differences between SARS-CoV and other coronaviruses are significant. The SARS-CoV genome has about 15 predicted open reading frames, six of which can be linked to other known coronavirus genes. These genes are: 1a-1b, spike (S), envelope (E), membrane (M) and nucleocapsid (N), which was found to be a multimeric form and to be involved in host cell signal transduction regulations [8,9]. The rest of the ORFs may encode genes with as yet unknown functions [1].

The development of antivirals for SARS-CoV has been vigorously pursued after the identification of the virus with mixed successes and challenges. Tan et al screened 19 clinically approved compounds for anti SARS-CoV activity, including nucleoside analogs, interferons, protease inhibitors, reverse transcriptase inhibitors and neuraminidase inhibitors. IFNs β-1b, α-n1, α-n3 and ribavarin showed anti-viral activities at high concentrations. However, significant cytotoxic effects or lack of efficacy were also observed [10]. For instance, in two other independent assays, ribavarin was shown to have little effect against SARS-CoV replication [11,12]. IFNs α, β-1b, α-n1 and α-n3 have also been tested for their anti SARS-CoV activities [10,12], however, the moderate inhibition effect of SARS-CoV replication by interferons could only be observed at very high concentrations [10,12]. As for new drug development, glycyrrhizin was reported to possess anti-SARS-CoV activity at high concentrations [12]. Clearly, continuing to search for potent anti-SARS-CoV compounds is absolutely necessary.

Unlike many other RNA viruses, coronaviruses synthesize multiple subgenomic mRNA fragments, with each subgenomic RNA usually encoding only one protein [4]. As a consequence, the transcription of coronavirus RNA is very important for virus replication. In this study, we studied the antiviral effects of ATA against SARS-CoV replication in Vero cells and found that ATA drastically inhibited virus replication by as much as 1000-fold compared to untreated controls, with little toxicity observed to be associated with ATA treatment. Anti-viral selectivity of ATA was demonstrated by its failure to inhibit adenovirus replication. Importantly, we found that ATA is a much more potent anti-SARS-CoV compound than IFNs α and β.

Vaccinia virus belongs to the Poxviridae family of double stranded DNA viruses, with a genome size of approximately 190 Kbp. Poxviruses are characterized by their complex structure and large genomes, permitting them a relatively high degree of independence from their host cells and the ability to synthesize close to 200 proteins. These viruses replicate in the cytoplasm of infected cells, in discrete locations termed viral factories, which have been shown to be free of host cell organelles (50). Since the virus does not enter the nucleus, it must bode for all proteins necessary for viral transcription and replication. Vaccinia virus encodes two protein kinases, F10L and B1R, as well as a protein phosphate, H1L. These three proteins perform essential functions in the virus lifecycle, suggesting that regulation of phosphorylation/dephosphorylation events play a key role during infection. Phosphorylation acts as a mediator of signaling pathways, activating or deactivating proteins involved in transducing signals. Viruses have evolved the ability to alter host signaling to create a favorable environment for their replication.

The vaccinia virus open reading frame designated H1L encodes a 19.3 Kdal dual specificity protein phosphatase that is expressed during the late stage of infection. This enzyme is transported into host cells at approximately 200 molecules per virus particle and a fraction of it is released into the cytoplasm upon uncoating (38, 47). The F10 kinase is also a virion component. This suggests that these proteins may have an immediate role in infection by regulating the phosphorylation state of specific proteins. The gene encoding H1L is well conserved amongst the Poxviruses, with homologues identified in variola virus, ectromelia virus, monkeypox virus, cowpoxvirus, myxoma virus and shope fibroma virus (38, 51). This enzyme shares the active site motif, HCXXXXXRS, common to the dual specific and protein tyrosine phosphatases (33, 34, 38). Two human homologues, termed VHR and VHX, also exist. H1L was the first phosphatase discovered with the ability to dephosphorylate both Tyr and Ser/Thr residues (37). A conserved Cysteine at position 110 within H1L has been shown to be essential for enzymatic activity towards both Tyr and Ser/Thr phosphorylated proteins. This suggests that the dephosphorylation reaction likely proceeds by a conserved mechanism in both cases (34).

H1L is essential for virus viability, as evidenced by the absence of viral gene transcription when the expression of H1L is repressed (47). Since the cascade-like nature of Poxvirus transcription relies upon early transcription to direct intermediate and ultimately late gene transcription (27), this block will prove fatal to the virus. Repression of H1L also leads to hyper-phosphorlyation of several viral proteins, including the products of the F18, A14 and A17 genes (35, 49). The function of the phosphate groups on these protein remains unknown. The exact role of H1L during infection has yet to be elucidated, but besides mediating viral transcription, the phosphatase has also been shown to alter host cell signaling pathways. Phosphorylated Stat1 can be dephosphorylated by H1L in vivo, blocking the expression of IFN-y induced genes (52). This may represent one means by which vv overcomes host defenses. In this study we demonstrate that ATA can inhibit the activity of H1L in vitro, and down-regulate the Erk signaling cascade. These events are proposed to be at least two of the ways that ATA exerts its anti-viral effect.

As will be seen, the aromatic polyanion Aurintricarboxylic Acid (ATA) has been shown to have a number of diverse activities although the mechanism by which ATA exhibits these effects is often poorly understood. It was initially postulated that ATA would inhibit the association of any nucleic acid binding protein with nucleic acid. Subsequent research has shown that nucleases appear to be more sensitive to inhibition by ATA than other enzymes.

Specifically, ATA has been shown to inhibit the RNA transcription of vesicular stomatitis virus [13]. It has alio been shown that ATA could interact with ribosomal proteins in vitro and inhibit protein synthesis [14,15]. ATA is also believed to promote cell survival and proliferation by activation of the IGF-IR signalling pathway (Beery et al., 2001, Endocrinology 142: 3098-3107) although it was previously believed that this activity was a result of ATA's inhibition of cellular endonucleases, discussed above.

ATA is also thought to inhibit transcription of iNOS genes (Tsi et al., 2002, Mol Pharmacol 101: 90-101) possibly by inhibiting upstream signal kinases.

Andrew et al. (1999, Immunopharmacology 41: 1-10) suggested that ATA at a concentration of 25 μM had effects on protein phosphorylation, in addition to inhibiting endonuclease activity.

Nakane et al. (1988, Eur. J. Biochem. 177: 91-96) showed that both Evans blue and ATA exhibited inhibitory effects on the in vitro activity of all DNA polymerases, including human DNA polymerases α, β, γ, DNA primase, calf-thymus terminal deoxynucleotidyltransferase, RLV reverse transcriptase, E. Coli DNA polymerase I and RNA polymerase; however, these compounds did not inhibit DNA, RNA or protein synthesis in intact cells at the concentrations which proved inhibitory in vitro, suggesting that the polymerases existed in an organized state in the nucleus, which protected them from these compounds. Similarly, Thompson and Reed (1005, Toxicol Lett 81: 141-149) showed that ATA inhibited a wide range of NAD(H)/NADP(H)-requiring enzymes in in vitro incubations using purified enzymes but the inhibitory effects were markedly reduced in incubations which more closely resembled a cellular milieu.

Cushman and Sherman (1992, Biochem Biophys Res Commun 185: 85-90) showed that ATA acted as an inhibitor of HIV-1 integration protein (IN). Similarly, Schols et al (1989, PNAS 86: 3322-3326) believed that ATA was targeting the CD4 receptor and thereby interfering with HIV infection. They further noted that ATA had no inhibitory effect at subtoxic concentrations for viruses that did not require the CD4 receptor to infect cells (herpes simplex virus, cytomegalovirus and vesicular stomatitis virus specifically were tested) indicating that “ATA is not a selective inhibitor of viruses other than HIV”.

Givens and Manly (NAR 3:405-418) tested the effect of ATA on RNA dependent DNA polymerases. It was believed that ATA was a nonspecific inhibitor of nucleotide-requiring enzymes in vitro but this same effect was not found in vivo. One would conclude that ATA would have a nonspecific inhibitory effect on all polymerases in a purified system, but no discernable effect under in vivo-like conditions. Furthermore, ATA was not tested against RNA dependent RNA polymerases, which is only found in RNA viruses.

It is of note that the literature lists several compounds which have been reported to be equivalent to ATA under certain conditions, including Evans Blue, suramin, and polyethylene sulfonic acid. In addition, Liang et al (JBC 278: 41734-41741) also lists a number of other compounds.

### SUMMARY OF THE INVENTION

According to a first aspect of the invention, there is provided a method of treating an individual infected with or suspected of being infected with an ATA-sensitive virus comprising administering to an individual in need of such treatment an effective amount of aurintricarboxylic acid (ATA) or a derivative thereof.

According to a second aspect of the invention, there is provided a method of identifying an organism inhibited by ATA comprising:

searching a protein structure database for a peptide of interest having a region homologous to R_{binding} region of SARS-CoV RdRp; and

determining if the homologous region contains catalytic residues for the protein of interest.

According to a third aspect of the invention, there is provided a method of screening organisms of interest for inhibition by ATA comprising:

incubating an organism of interest under appropriate growth conditions in the presence of ATA; and

determining if growth of the organism of interest has been inhibited.

According to a fourth aspect of the invention, there is provided a method of inhibiting growth of an organism comprising administering an effective amount of ATA or a derivative thereof, wherein the organism comprises an essential protein having a region homologous to R_{binding} region of SARS CoV RdRp, wherein said homologous region comprises at least one catalytic residue of the protein.

According to a fifth aspect of the invention, there is provided a method of preparing a medicament for treating an individual infected with or suspected of being infected with an ATA-sensitive virus comprising combining an effective amount of aurintricarboxylic acid (ATA) and a suitable excipient.

### BRIEF DESCRIPTION OF THE DRAWINGS

FIG. 1. Vero cells were infected with SARS-CoV and treated with dilutions of aurintricarboylic acid. At 24 h. post infection, supernatant samples were harvested for a plaque assay. The virus titres of ATA treated and untreated samples were calculation and represented by plaque-forming unit (PFU/ml). The experiments were repeated at least 3 times, with SD being approximately 10%.

FIG. 2. Vero cells were infected with SARS-CoV and treated with serially diluted concentrations of aurintrucarboxylic acid. After 24 h., cells were harvested and subjected to 4-12% SDS-PAGE; protein samples were subsequently transferred to PVDF membrane and probed with a mouse monoclonal antibody against SARS-CoV spike protein and anti-α-actinin antibodies. A rabbit anti-mouse antibody conjugated with horseradish peroxidase was used as the secondary antibody. The blot was subsequently developed with SuperSignal West Femto Western Blot kit (Pierce, Rockford, Ill.). FIG. 2a shows inhibition of SARS-CoV replication without the pre-treatment of Vero cells; FIG. 2b shows inhibition effect of the same inhibitors in 2a with the pre-treatment of Vero cells for 12 h. at 37° C.

FIG. 3. Vero cells were infected with SARS-CoV and treated with serially diluted concentrations of aurintrucarboxylic acid. HEK293 cells were transfected with an adenovirus construct expressing EGFP. After 24 h., cells were harvested and subjected to 4-12% SDS-PAGE; protein samples were subsequently transferred to PVDF membrane and probed with a mouse monoclonal antibody against SARS-CoV spike protein (for Vero cell extracts), mouse monoclonal antibody against EGFP (for HEK293 cell extracts) and anti-α-actinin antibodies. A rabbit anti-mouse antibody conjugated with horseradish peroxidase was used as the secondary antibody. The blot was subsequently developed with SuperSignal West Femto Western Blot kit (Pierce, Rockford, Ill.).

FIG. 4A. Sequence homology between RDRP, Calpain and Yersina proteins. Frames show putative ATA target sites.

FIG. 4B. Evolutionary associated, sequence similarity of 1QZ0 (1) SARs RSRP (2) and M-Calpain (3).

FIG. 5. Structural alignment between proteins from SARS-CoV and other RNA viruses. The structural alignment between proteins from SARS-CoV and other RNA viruses based on their 3D atomic coordinate files were performed using Dali and LGA with manual alignment. Solvent inaccessible residues were represented in upper case, while the solvent accessible residues are represented in lower case. The residues that have hydrogen bond to main chain amide are in bold and residues that have hydrogen bond to main chain carbonyl are underlined, Finally, residues that are joined by disulphide bonds are represented in cedilla. 1C2PA—Hepatitis C virus RNA-dependent RNA polymerase; 1DF0A—m-calpain; 1hhsa—bacteriophage phi6 RNA-dependent RNA polymerase; 1KHVA—rabbit hemorrhagic disease virus RNA-dependent RNA polymerase; 1O5SA—SARS-CoV RNA-dependent RNA polymerase; 1QZ0A—Yersinia Pestis phosphatase yoph; 1RDR0—poliovirus RNA-dependent RNA polymerase; 1S4FA—bovine viral diarrhea virus RNA-dependent RNA polymerase; 1SH0A—Norwalk virus RNA-dependent RNA polymerase; 3HVTA—HIV Type 1 RNA-dependent RNA polymerase.

FIG. 6. Calculated structure (using Autodock) for the interaction of ATA with ypoH. YpoH is a protein tyrosine phosphatase which is essential for virulence in Yersinia pestis. It is known that the functionality of this protein is inhibited strongly by ATA. This figure shows the ten most possible confirmations of ypoh-ATA complexes. The border residues that have contact with ATA are shown and are part of a region that is structurally conserved between ypoh, m-captain and SARS-CoV RdRps, and are constituted mainly of anti-parallel β-strand-turn-β-strand hairpin structures. The two dimensional chemical structure for ATA (C₂₂H₁₄O₉) is shown below.

FIG. 7. Calculated structure (using Autodock) for the interaction of ATA with m-calpain. The neutral protease (calpain) is a class of cytosolic enzyme that is activated during apoptosis. It is known to be inhibited strongly by ATA. This figure shows the ten most possible confirmations of m-caplain-ATA complexes. The border residues that have contact with ATA are shown and are in a region that is structurally conserved between ypoh, m-caplain and SARS-CoV RdRps and are constituted mainly of anti-parallel β-strand-turn-β-strand hairpin structures.

FIG. 8. Calculated structure (using Autodock) for the interaction of ATA with RdRp of SARS-CoV.

FIG. 9. Structure of ATA.

FIG. 10 is a graph showing dynamics of West Nile Virus Replication.

FIG. 11 is a graph showing inhibition of West Nile Virus replication by ATA at increasing concentrations of ATA.

FIG. 12. Inhibition of vaccinia virus replication by ATA. Hela cells infected with vv (WR) and treated with dilutions of ATA. Plaque counts were done in triplicate on BSC-1 cells, using a 200 μl inoculum and checked at 24 hours post-infection.

FIG. 13. Time-course inhibition of vaccinia virus replication by ATA. RK13 cells were infected with vv(WR) and the virus titre was checked at various time points. Plaque counts were done in triplicate on BSC-1 cells, using a 200 ul inoculum and checked at 24 hours post-infection.

FIG. 14. Silver stained gel of the 46 Kdal GST-H1L fusion protein purification. Lane 1: protein mass markers, lane 2: GST column flow through, Lane 3: GST fraction A7 Lane 4: QFF fraction A5, Lane 5: QFF fraction A7, Lane 6: QFF fraction A9.

FIG. 15. Effect of ATA on H1L catalyzed hydrolysis of pNPP. The reactions were performed at 37 C at 0.06, 0.125, 0.25, 0.5, 1, 2 and 4 mM pNPP. All reactions were performed in triplicate.

FIG. 16. IC50 analysis of H1L inhibition. Reactions were performed at a substrate concentration equal to the Km value (1 mM), at 37 C. All reactions were performed in triplicate.

FIG. 17. Comparative inhibition of phosphatase activity by ATA. Activities of each enzyme were standardized, with the same amount of activity being used in the reactions. All reactions were done at 37 C, in serial dilutions of ATA.

Table 1. Vero cells were seeded in a 96-well plate. Dilutions of ATA, interferons α and β were added. After 24 h. 50 μl of Reaction Solution from XTT kit was added to each well and incubated at 37 C for 4 h. Activities of cell proliferations were reflected by spectrophotometric readings. The concentrations of each reagent that inhibits 50% cell proliferation activities (CC₅₀) were compared with the concentrations that inhibit 50% of SARS-CoV replication (EC₅₀), and designated as the selection index (SI).

Table 2. Estimated free energy (final intermolecular energy+torsional free energy) of different targeted proteins in complex with ATA using Autodock.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “effective amount” refers to an amount that is sufficient to achieve the desired result. In regards ATA, an effective amount is capable of inhibiting the target organism.

As used herein, “inhibition” in all its grammatical forms does not imply a complete cessation but rather indicates the activity being inhibited occurs at a lower rate or efficiency.

Described herein is a method of inhibiting replication of an organism which has an essential enzyme which includes a binding groove that is bound by ATA comprising administering to a patient in need of such treatment an effective amount of aurintricarboxylic acid (ATA).

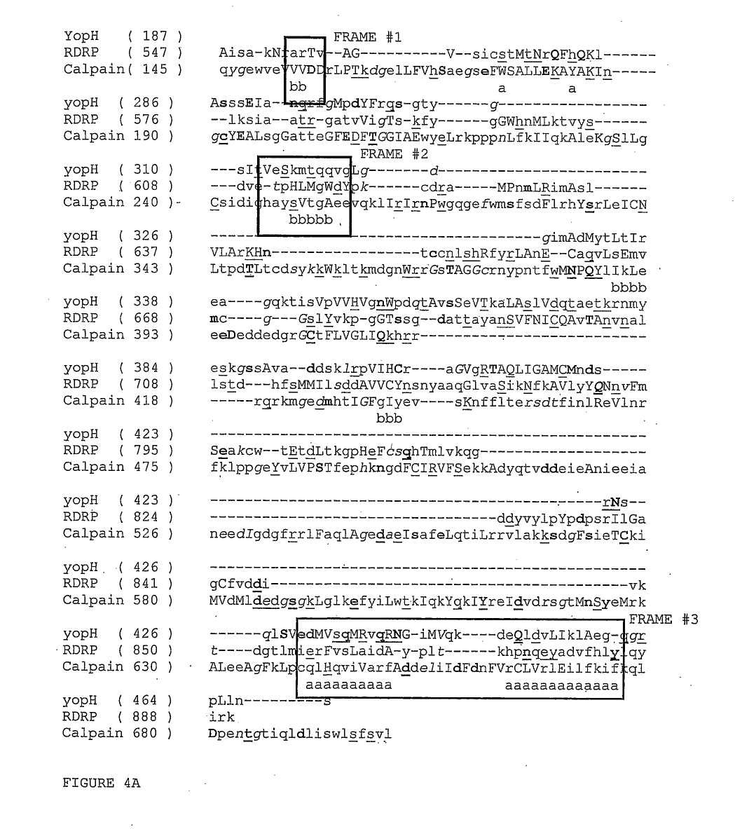

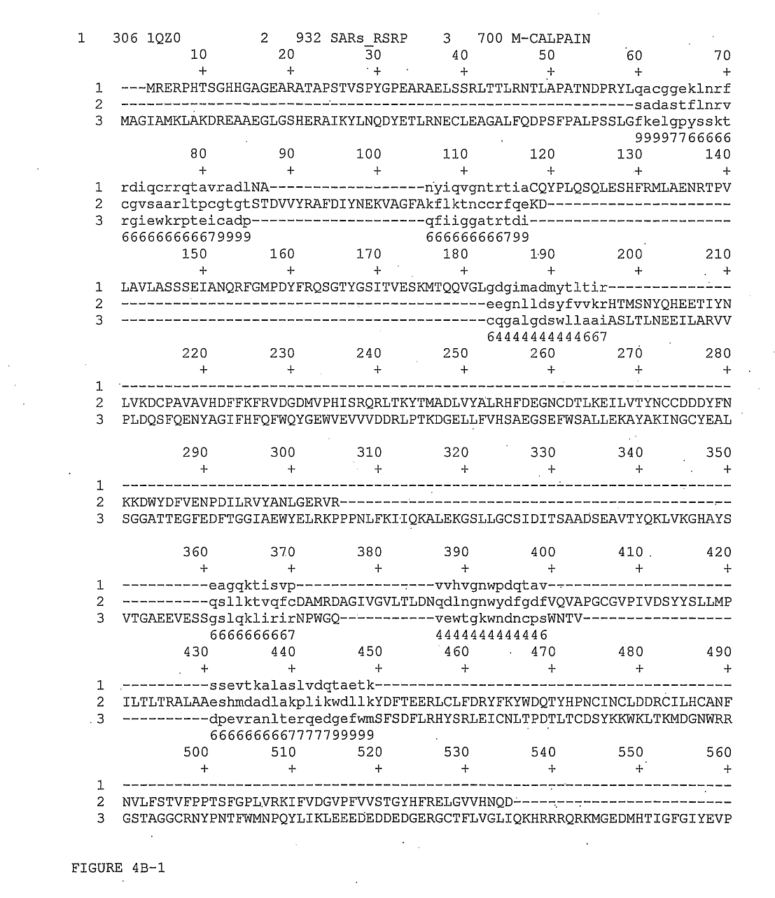

As can be seen in FIG. 4, a putative 3D structure of SARS RNAP and ATA (using an ATA ligand model) shows the template binding groove of RNAP which is also bound by ATA. That is, ATA binding at this groove prevents template binding and therefore viral replication by RNAP.

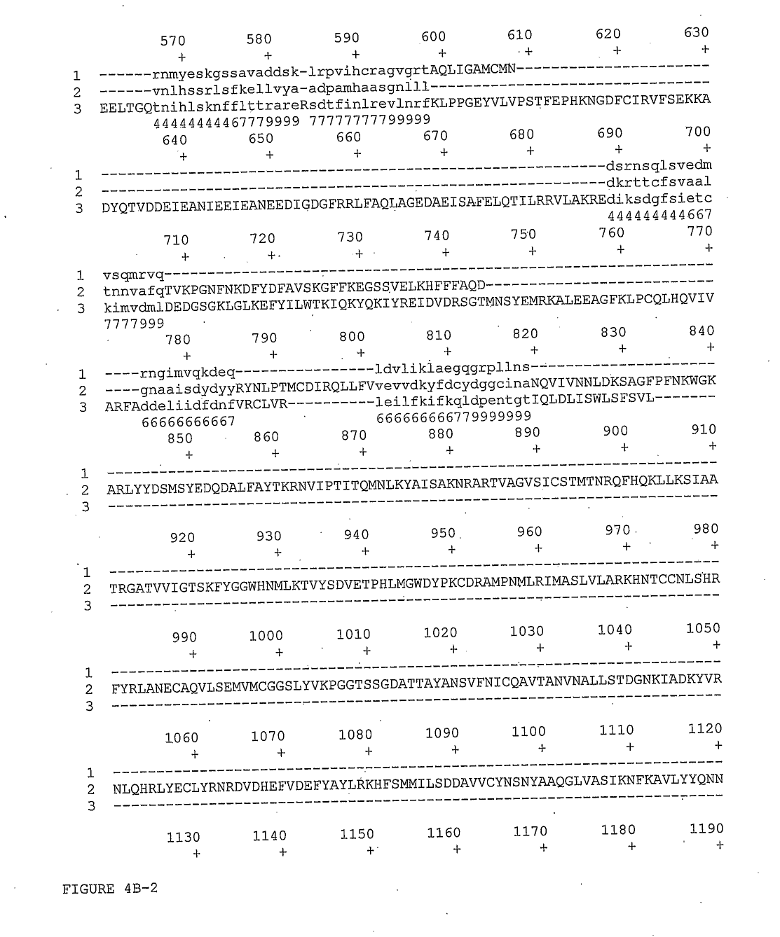

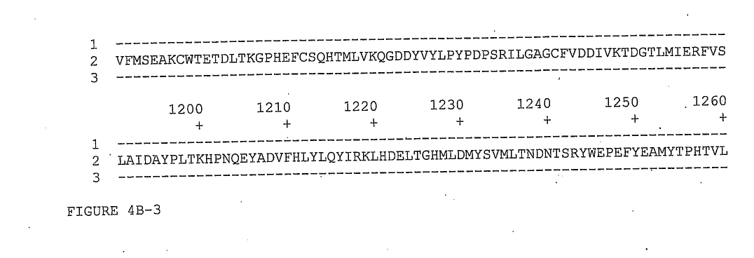

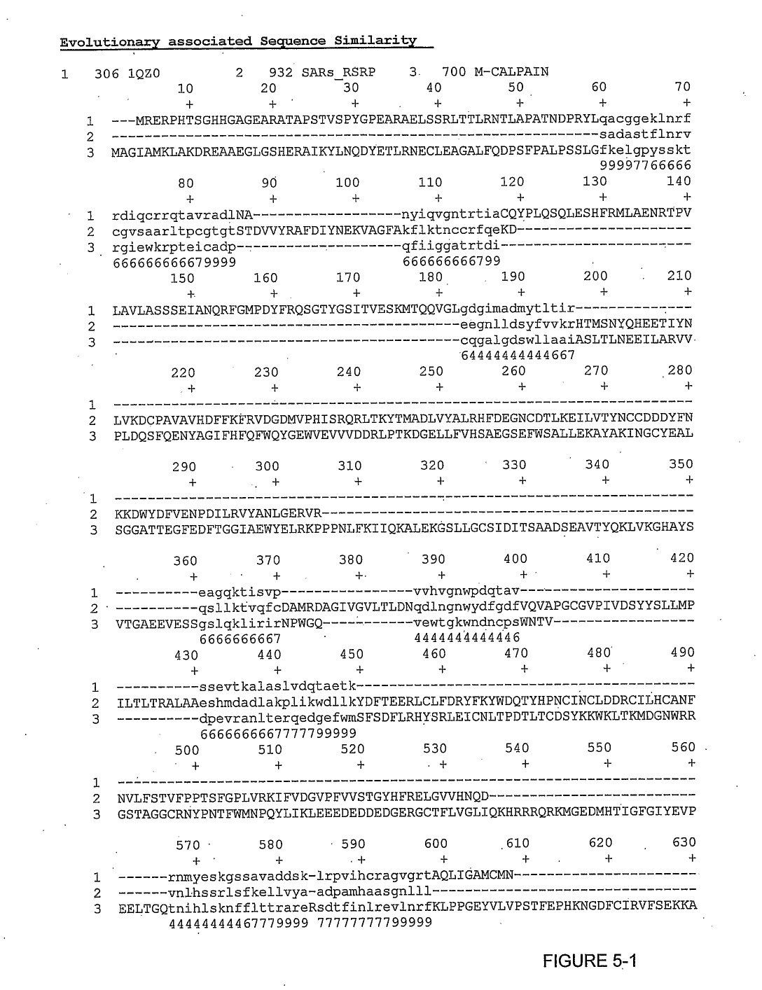

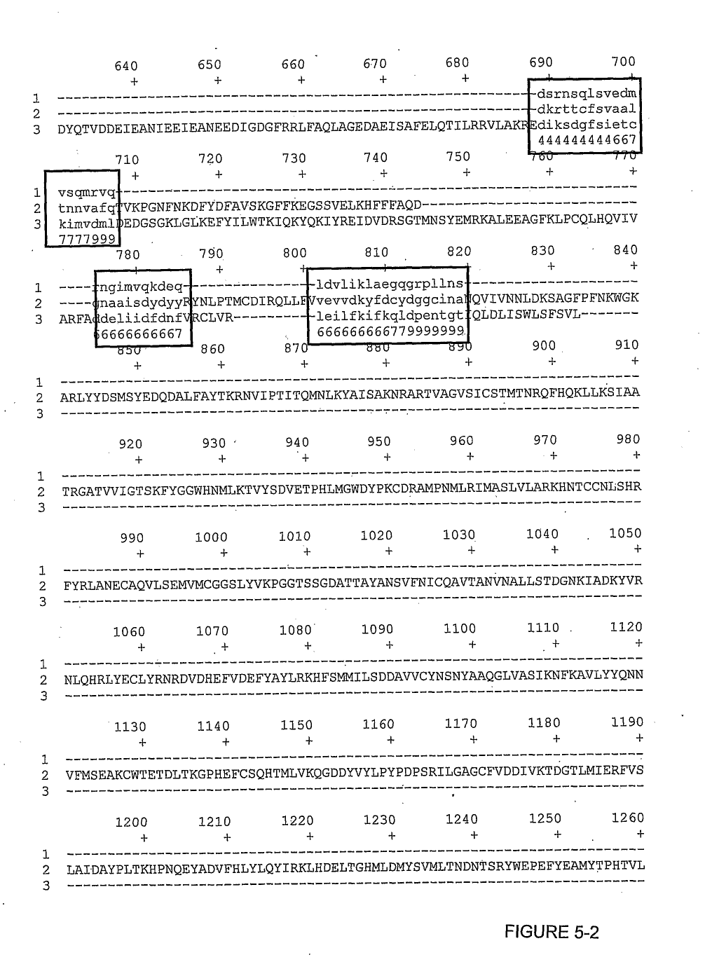

Furthermore, as can be seen from the sequence comparison shown in FIG. 5, enzymes from other organisms known to be inhibited by ATA have similar grooves. It is important to note however that as discussed above ATA has many putative functions and it is possible that it acts through a number of different mechanisms in, addition to the groove binding activity discussed above.

As discussed below, ATA is an effective anti-viral for coronaviruses, for example, SARS-CoV, West Nile Virus, Norwalk, Dengue and Japanese Encephalitis virus. However, we have tried ATA on Ebola and Adenovirus and saw no effect.

In addition, the inhibitory effect of aurintricarboxylic acid (ATA) on vaccinia virus replication in tissue culture is described herein. Concentrations of ATA in the range of 400 ug/mL decreased viral replication as much as 250,000 fold as compared to controls. A block in replication was evident at drug concentrations as low as 25 μg/mL. Inhibition of the viral phosphatase, H1L, which is essential for virus replication, was found to be one mechanism through which ATA exerts its anti-viral effect. The IC50 value of ATA against H1L was found to be 16 μM. This block in enzyme activity leads to a global shutdown in viral protein production and subsequent viral replication. Western blotting also revealed that the ERK signaling cascade was down-regulated in cells treated with ATA. The activity of the ERK signaling cascade has previously been implicated in the vaccinia virus lifecycle. As discussed above, H1L is highly conserved among related viruses and as such ATA is also an effective treatment for infections caused by variola virus, ectromelia virus, monkeypox virus, cowpox virus, myxoma virus and shope fibroma virus.

As discussed above, ATA is a poly-aromatic carboxylic acid derivative, displaying a wide array of biological activities. It has been shown to inhibit endonucleases, topoisomersaes, kinases and phosphatases, as well as apoptosis (26, 48). It has the ability to stimulate the activity of the Insulin-like Growth Factor 1 and Jak2-Stat5 signaling pathways (53). Small molecules like ATA have been shown to interact with receptors at the cell surface (55), where they may alter the activity of signaling pathways. Previous studies have shown that ATA inhibits PTP activity (32, 55).

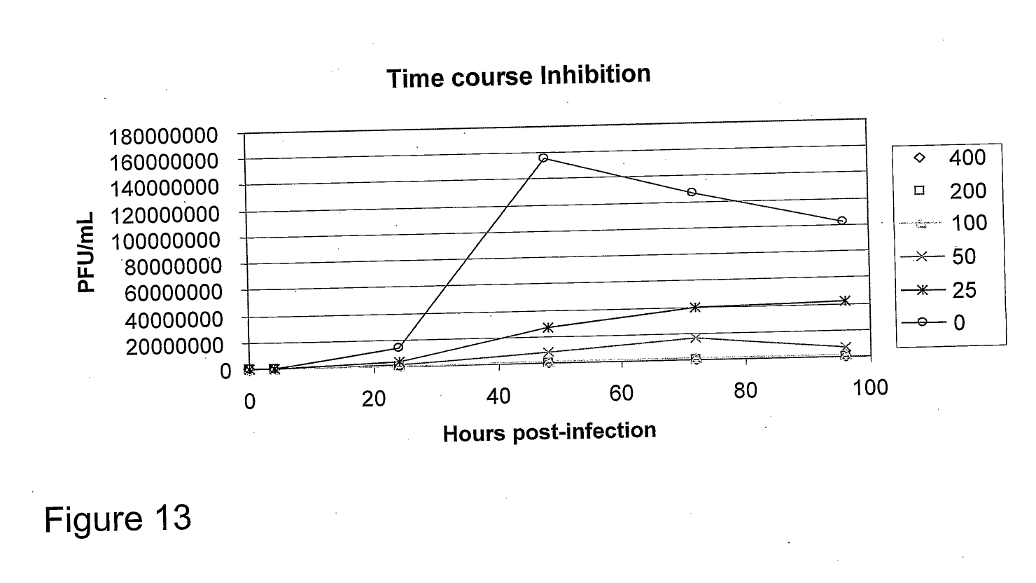

As discussed herein, ATA inhibited the replication of SARS-Cov. Specifically, when used at concentrations of 800 μg/mL, a 1000 fold decrease in replication was observed, while 400 μg/mL dropped replication 100 fold. Furthermore, when used at 400 μg/mL, ATA could inhibit vaccinia virus replication 250,000 fold, dropping the level of recoverable virus to near non-detectable levels. (FIG. 13). This inhibition was seen in all cell lines tested, including Hela, RK13, Huh7 and vero cells. The virus titre peaked at close to 48 hours post-infection in the untreated controls. This time point also showed the greatest difference in virus titre between treated and untreated cells. Inhibition was seen still seen at ATA concentrations as low as 25 μg/mL. To determine whether ATA has a general antiviral effect, it was tested against Adenovirus. In this case, the drug actually stimulated viral replication. Viral replication was seven times higher when ATA was present compared to no ATA.

The vaccinia virus dual-specific PTP is essential to virus viability. Experiments have shown that vaccina virus mutants lacking a functional copy of H1L have reduced levels of transcription, leading to a resulting lack of replication and infectivity (47). RT-PCR experiments were used to detect this block in viral transcription. The presence of F10 mRNA was decreased in cells treated with ATA. This is consistent with a block in viral transcription. As a control, the mRNA level of GADPH was also followed. This is a host cell house-keeping gene, and a commonly used control. GADPH levels increased in infected cells treated with ATA. This is as expected because during a productive infection, the virus shuts down host protein synthesis. If ATA were inhibiting the virus, you would expect GADPH levels to increase compared to non-treated (ATA) cells.

The homologue of H1L found in other poxviruses has also been shown to be essential (38, 51). H1L has also been implicated in subverting host defense mechanisms in order to allow for a productive infection. Studies have demonstrated that infection of cells with vaccinia virus leads to a reduction in gamma interferon signal transduction. This was found to be a direct result of lower levels of phosphorylated Stat1 in vv infected cells. The phosphorylation of Stat1 is required for its signal transducing activity. Levels of H1L present in the cells correlated inversely with the levels of phosphorylated Stat1. This provides strong evidence that Stat1 is a bona fide substrate for H1L in vivo (52). The gamma interferon signal transduction cascade induces the expression of genes involved in the anti-viral response. This includes the expression of major histocompatibilty complex (MHC) on antigen presenting cells and the activation of natural killer cells (43). The dephosphorylation of Stat1 by H1L may represent one mechanism by which vaccinia virus evades host immune responses.

H1L has also been implicated in other processes that are important for host cell function. Alternative splicing of RNA represents a means by which cells produce diverse and structurally distinct sets of proteins. The SR proteins are responsible for regulating this activity, and phosphorylation in the RS domain has been shown to be essential for proper functioning. When extracts of vaccina virus infected cells were blotted with antibodies against SR proteins, it was found that they were hypo-phosphorylated. Further experiments involved incubating purified SR proteins with purified H1L in an in vitro dephosphorylation assay. This resulted in dephosphorylation of the SR proteins in the RS domain, leading to their inactivation as splicing factors (41).

Several vaccina virus proteins are also substrates for H1L. The A17 and A14 phospho-proteins of vaccinia virus are implicated as being essential viral membrane components. The phosphorylation state of these proteins is regulated by the F10 kinase and H1L. Repression of both A14 and A17 leads to aberrant morphogenesis. However, the role of the phosphorylation of these proteins remains unclear, although it may regulate binding interactions and glycosylation levels (35, 49).

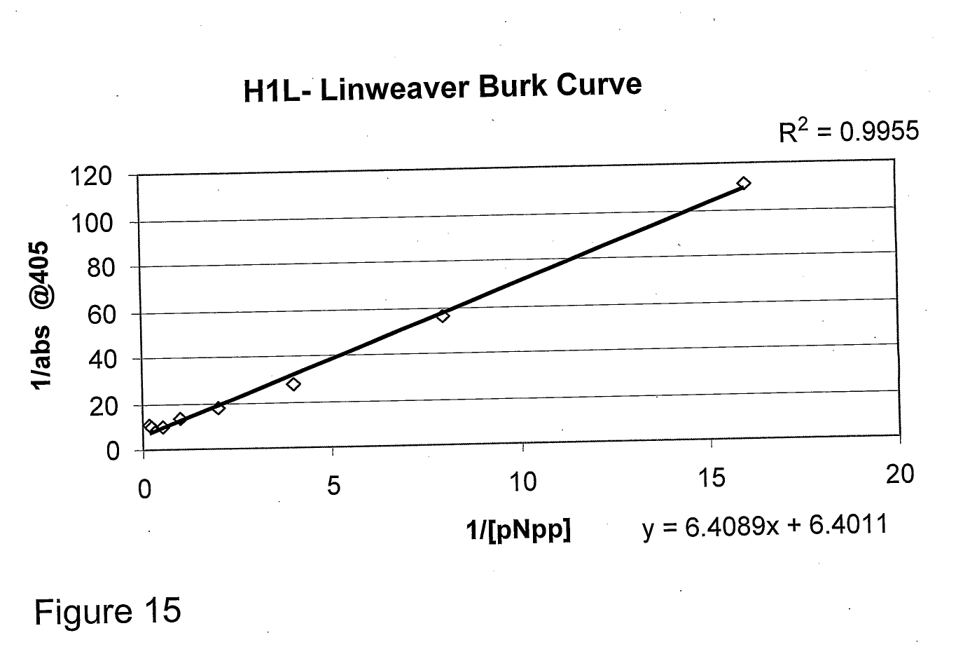

This report is the first to describe kinetic parameters for H1L (FIG. 15). These values are specific to the reaction conditions and the substrate used. Thus, when reactions are performed at a different temperature, pH, or enzyme concentration, the result will vary. Also, the use of a different substrate would produce different results because the affinity of the phosphatase is different for any substrate. Two very important kinetic values for any enzyme are the Vmax and Km values. The Vmax is defined as the maximum rate of an enzyme catalyzed reaction when the active site is saturated with substrate (45). The Vmax for H1L was determined to be 6.40 (abs. units). This value is a reflection of the absorbance reading, and thus, the reaction rate. The Km is defined as the substrate concentration at which half maximal velocity is reached (45). The Km value for H1L was found to be 1 mM pNPP. This means that when pNPP is used at a concentration of 1 mM, the reaction rate will be 50% of the maximum value. The Vmax of H1L was reached at substrate concentrations of 4 mM. These values make allow for determination of the potency of ATA towards H1L.

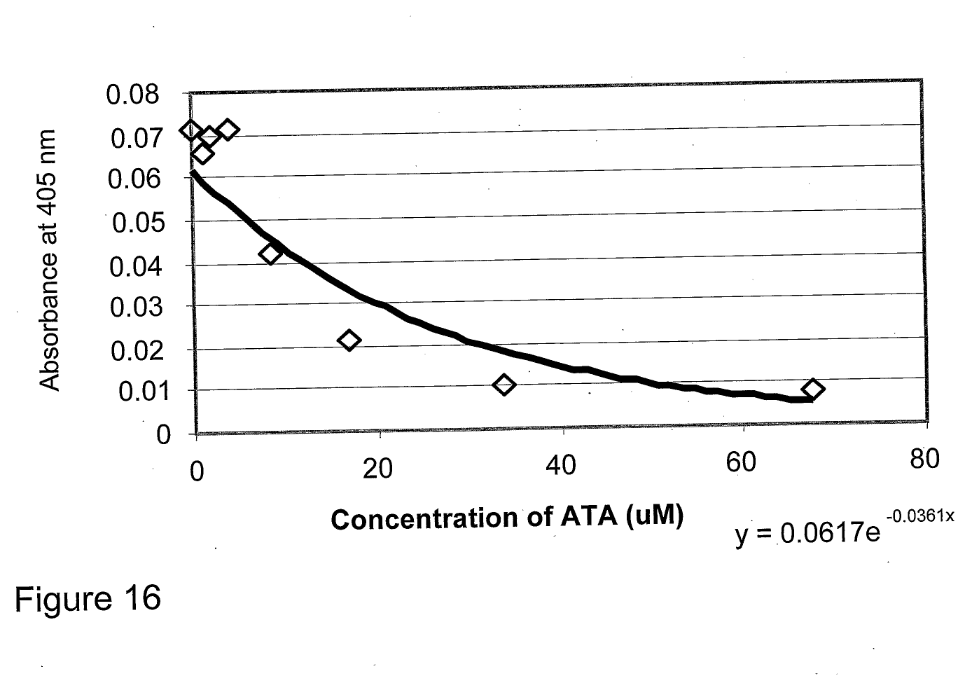

Phosphatase activity assays were employed to determine if ATA could inhibit H1L. It was found that ATA could block H1L dependant dephosphorylation of pNPP, a commonly used experimental substrate. The IC50 value for this inhibition was 16 μM (FIG. 16). This means that at an ATA concentration of 16 μM, the activity of H1L is reduced by 50%. The control reactions using GST-A30L rule out the possibility of a contaminant phosphatase in the preparation. It also shows that the GST tag does not confer phosphatase activity. ATA had a greater inhibitory effect on the T-cell phosphatase than on H1L, while LAR was less affected. This would mean that using ATA at a concentration effective against H1L would also affect the activity of T-cell PTP in host cells. These assays only detect phosphatase activity towards tyrosine residues. Further experiments need to be done to determine if ATA can also inhibit the Ser/Thr activity of H1L. It should also be noted that the IC50 value against ATA is much lower than the concentration required to inhibit 50% growth in tissue culture. It may be that not all the drug enters the cell, thus decreasing its intracellular concentration. Also, ATA is known to bind to proteins and DNA, decreasing the amount of drug available to inhibit the phosphatase.

Inhibition of H1L will result in a defective infection. Virus transcription will be blocked, to a level proportional to the inhibition of H1L. This means that there will be a reduction in the production of all viral proteins, including H1L itself. This would obviously have a detrimental effect on the virus as proteins necessary for replication, transcription, viral morphogenesis and immune evasion would be found at significantly reduced levels. Host cell gamma-interferon signaling would be restored to wild-type activity, leading to an anti-viral response. H1L inhibition also leads to the hyper-phosphorylation of several viral and host proteins. Thus, the regulatory function of the phosphate groups on these proteins would be lost.

A further aspect of this project was to determine the effect, if any, that ATA exerts on host cell signaling pathways. Vaccinia virus encodes two kinases, B1R and F10L, as well as a phosphatase, H1L. Phosphorylation events are key mediators of host cell signaling cascades, and it seems likely that vaccinia virus exploits these cascades for its own good. Viral interference with host cell signaling is well documented (36, 39, 52, 54). The MAPK (mitogen activated protein kinase) cascade is responsible for controlling cell growth and proliferation, and responses to stress. One route of this cascade, controlled by the extra-cellular regulated kinase 1/2 (Erk1/2), has been shown to be essential for a productive vv infection (57).

Western blotting experiments using an anti-phospho Erk1/2 antibody showed that ATA treatment of cells lead to a decrease in the levels of phospho-Erk protein. Similar results were obtained for the JNK and p38 MAPKs. This phosphorylation is necessary for further transduction throughout the pathway. Thus, this may represent another mechanism by which ATA inhibits vaccinia virus. It is not known, however, at which point in the signaling cascade this block appears at. Inhibition of any kinases upstream of Erk would lead to a similar result. Interestingly, VHR, a human phosphatase related to H1L, as been shown to dephosphorylate Erk1/2 (24, 56). It is possible that H1L may also perform this function, however it would not be expected to lead to complete Erk1/2 dephosphorylation in vivo, as this would block viral infection. It is likely that a balance exists in the phosphorylation state of signaling proteins. Vaccinia virus likely alters this balance, by way of its kinases and phosphatase, to create an intracellular environment more amenable to its own replication.

This study provides evidence that ATA is of therapeutic use against Poxviruses. We have shown that ATA is able to inhibit the phosphatase activity of H1L, with an IC50 value of 16 μM. This enzyme is conserved throughout the Poxviruses, and thus they are all likely to be susceptible to this drug. This enzyme plays essential roles in many aspects of the viral lifecycle, including viral transcription and immune evasion. This makes ATA an ideal drug candidate, as resistance to the drug would difficult to gain. Finally, ATA is able to down-regulate Erk mediated signaling, which is required for vv infection.

As will be appreciated by one of skill in the art, other possible ATA targets can be identified by several different means. For example, the knowledge of the consensus sequence for an ATA binding domain means that sequence and/or structure databases can be searched for enzymes with a similar binding groove. Thus, one aspect of the invention is directed to a method of identifying ATA-target organisms comprising screening a suitable database for matches to a consensus sequence for ATA binding domain, thereby identifying an ATA-inhibitable organism. Another aspect of the invention is directed to organisms identified by this search.

Specifically, protein structural studies can be carried out to investigate the potential binding modes/sites of ATA onto RNA-dependent-RNA-polymerases (RdRp) from SARS-CoV and other pathogenic positive-strand RNA viruses, as well as other proteins in SARS-CoV based on the fact that ATA binds to Ca²⁺-activated neural protease (m-calpain), the protein tyrosine phosphatase (PTP) and HIV integrase which have existing crystal structures. Eight regions with homologous 3D-confirmation were derived from ten proteins of interest. One of the regions, R_{binding} (754-766 in SARS-CoV's RdRp), located in the palm sub-domain consists of mainly anti-parallel β-strand-turn-β-strand hairpin structures that include two of the three RdRp catalytic sites (Asp 706, Asp 761), was also predicted by a molecular docking method (based on free energy binding of ΔG) to be an important binding motif recognized by ATA. The existence of this strictly conserved region that includes catalytic residues, coupled with the homologous ATA binding pockets and their consistent ΔG values indicates that ATA is involved in an analogous inhibition mechanism in SARS-CoV's RdRp, m-calpain, PTP and HIV integrase. Furthermore, as discussed below, other ATA-inhibited organisms can be identified using the searching method described herein. In another aspect, the invention is directed to this method of identifying organisms having similar motifs in key enzymes as well as the use of ATA to inhibit growth of the organisms so identified.

Alternatively, ATA-sensitive organisms can be determined by growing an organism under normally reproductive conditions in the presence of ATA and then determining if reproduction has occurred. This may be done by comparison with an untreated control. For example, if the organism is a bacteria or fungus, the organism may be grown in culture media at an appropriate incubation temperature. If the organism of interest is a virus, the organism is grown under appropriate culture conditions in the presence of ATA, virus is harvested and viral numbers are determined, for example, by a plaque assay.

According to another aspect of the invention, there is provided a method of inhibiting replication of an organism having an essential enzyme having an ATA binding domain comprising administering to that organism an effective amount of ATA.

As used herein, “aurintricarboxylic acid derivatives” or “ATA derivatives” includes chemically modified variants thereof, that is, compounds having the same general structure as ATA and further having the same biological activity as ATA and pharmaceutically acceptable salts thereof, wherein “biological activity” refers to the inhibition of viral replication.

In a preferred embodiment, ATA is used as a pharmaceutical composition for treating a patient suffering from or at risk of developing or suspected of having a coronavirus infection. Administration of an effective amount of ATA will have at least one of the following effects: inhibition of RNA synthesis, reduction of viral load, and amelioration of associated symptoms.

As will be appreciated by one of skill in the art, members of the coronavirus family include but are by no means limited to Bovine viral diarrhoea virus, feline infectious peritonitis, Porcine epidemic diarrhea virus, mouse hepatitis virus, human coronavirus 229E and human coronavirus OC43.

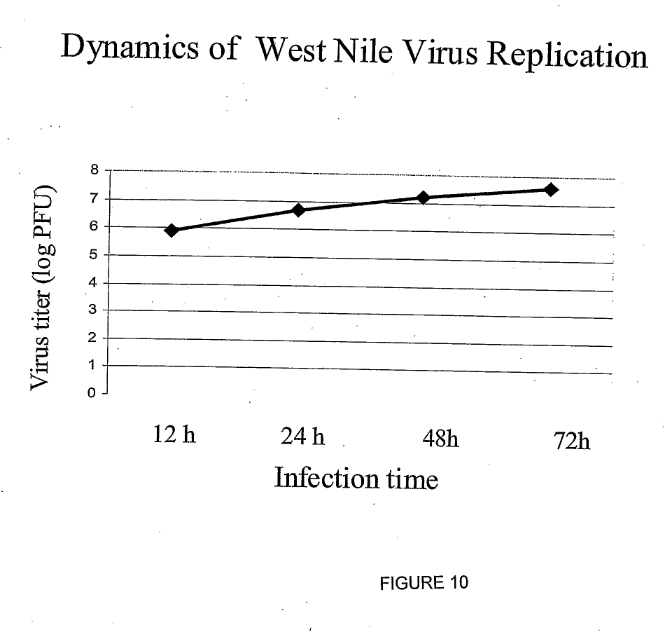

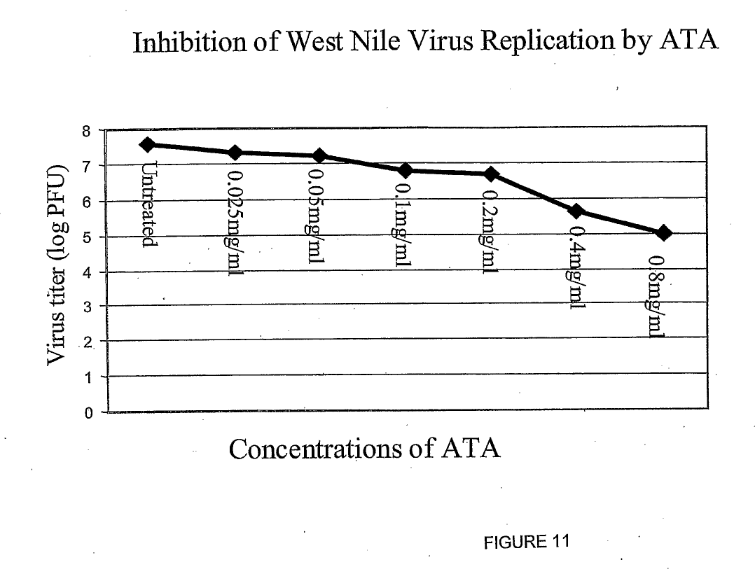

In another embodiment, ATA is used as a pharmaceutical composition for treating a patient suffering from or at risk of developing or suspected of having a West Nile Virus infection, a Norwalk virus infection, a Dengue virus infection, or a Japanese Encephalitis infection. Administration of an effective amount of ATA will have at least one of the following effects: inhibition of RNA synthesis, reduction of viral load, and amelioration of associated symptoms. Specifically, ATA has been shown to inhibit West Nile Virus 10000 fold as can be seen in FIGS. 10 and 11.

In a preferred embodiment, ATA is used as a pharmaceutical composition for treating a patient suffering from or at risk of developing or suspected of having a poxvirus infection. Administration of an effective amount of ATA will have at least one of the following effects: inhibition of RNA synthesis, reduction of viral load, and amelioration of associated symptoms. As discussed above, members of the poxvirus family include but are by no means limited to variola virus, ectromelia virus, monkey pox virus, cowpox virus, myxoma virus and shope fibroma virus.

It is of note that ATA discussed above may be prepared to be administered in a variety of ways, for example, topically, orally, intravenously, intramuscularly, subcutaneously, intraperitoneally, intranasally or by local or systemic intravascular infusion using means known in the art and as discussed below.

It is of note that as discussed herein, the ATA may be arranged to be delivered at a dosage of about 25 mg to about 800 mg per kg of the subject, or about 50 mg to about 800 mg per kg of the subject, or about 100 mg to about 800 mg per kg of the subject or about 200 mg to about 800 mg per kg of the subject or about 400 mg to about 800 mg per kg of the subject. As will be apparent to one knowledgeable in the art, the total dosage will vary according to the weight of the individual as well as other factors, for example, the age and condition of the patient. As discussed above, in some embodiments, an effective amount of ATA may be an effective concentration of between 10-800 μg/ml, between 50-800 μg/ml, between 100-800 μg/ml or between 400-800 μg/ml.

In some embodiments, ATA at concentrations or dosages discussed above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

The invention will now be described by examples; however, the invention is not limited to the examples.

### Cell Culture and Viral Plaque Assay

The African green monkey kidney cell line Vero cells were cultured in Dulbecco's Modified Eagle's Medium, supplemented with 10% heat-inactivated fetal bovine serum (Inivitrogen, Carlsbad Calif.), 1% penicillin/streptomycin and 10 mM HEPES (pH=7.2). Vero cells have been shown to be susceptible to SARS-CoV infection [4]. All cell cultures were maintained in a humidified 5% CO₂ incubator at 37° C. In all experiments, the multiplicity of infection (MOI) was 0.01. Aurintrycarboxylic acid (Sigma, St. Louis, Mo.) was prepared in the culture media and added into the samples in serial dilutions comprised of 0.8 mg/ml, 0.4 mg/ml, 0.2 mg/ml 0.1 mg/ml and 0.05 mg/ml. Plaque assays were performed 24 h. post infection using procedures as described [16]. We also performed inhibition analysis with interferon α and β both at the concentration of 5000 IU/ml [10,12].

### Western Blotting

Protein samples from Vero cell extracts were fractionated on 4-12% SDS-PAGE (Invitrogen Carlsbad Calif.) and transferred to PVDF membrane using a semi-dry protein transfer apparatus (Bio-Rad, Hercules Calif.). The membrane was blocked for 1 hr with 5% skim milk in TBS buffer (20 mM Tris base, 137 mM NaCl, pH7.6) containing 0.2% Tween-20. The membrane was then probed with a mouse monoclonal antibody against SARS-CoV spike protein. Rabbit anti-mouse HRP-conjugated antibody (Amershambiosciences, Piscataway, N.J.) was subsequently added for an additional incubation of 1 hr at 37° C. The results were revealed using Pierce Biotechonlogy SuperSignal West Femto Maximum Sensitivity Substrate (Rockford, Ill.).

### Real-Time RT-PCR Analysis

The analysis was performed with Prism 7700 real-time PCR instrument from PerkinElmer (Wellesley, Mass.) following the manufacturer's protocol. Supernatant samples from SARS-CoV infected Vero cells were collected for viral RNA extractions using RNeasy kit from Qiagen (Valencia, Calif.). Primers and probe used in the RT-PCR are as follows: Probe: 6FAM-ACCCCAAGG TTTACCC (SEQ ID No. 1); Forward: ACCAGAATGGAGGACGCAATG (SEQ ID No. 2); and Reverse: GCTGTGAAC CAAGAC GCAGTATTA T (SEQ ID No. 3).

### Inhibition of Adenovirus Expressing EGFP

ATA was used in a comparative study for inhibition of the adenovirus Adeno X, which carries a replication reporter gene expressing EGFP (Clontech, Palo Alto, Calif.). Approximately 5 mg of adenovirus X were transfected into HEK293 cells, using Effectene (Qiangen, Valencia, Calif.), followed by addition of serial dilutions of ATA-Western blot analysis was followed using the antibody against EGFP (Clontech, Palo Alto, Calif.).

### Cell Proliferation Assay

The XTT kit (Roche, Mannheim, Germany) was used to measure the toxicity of ATA. Briefly, Vero cells were seeded in a 96-well plate, dilutions of ATA and interferons were added to the cells and incubated for 24 h. The colorimetric detection reagent from the XTT kit was subsequently added to the cells. Results were determined by spectrophotometer at 450 nm.

### Inhibition of SARS-CoV Replication by ATA

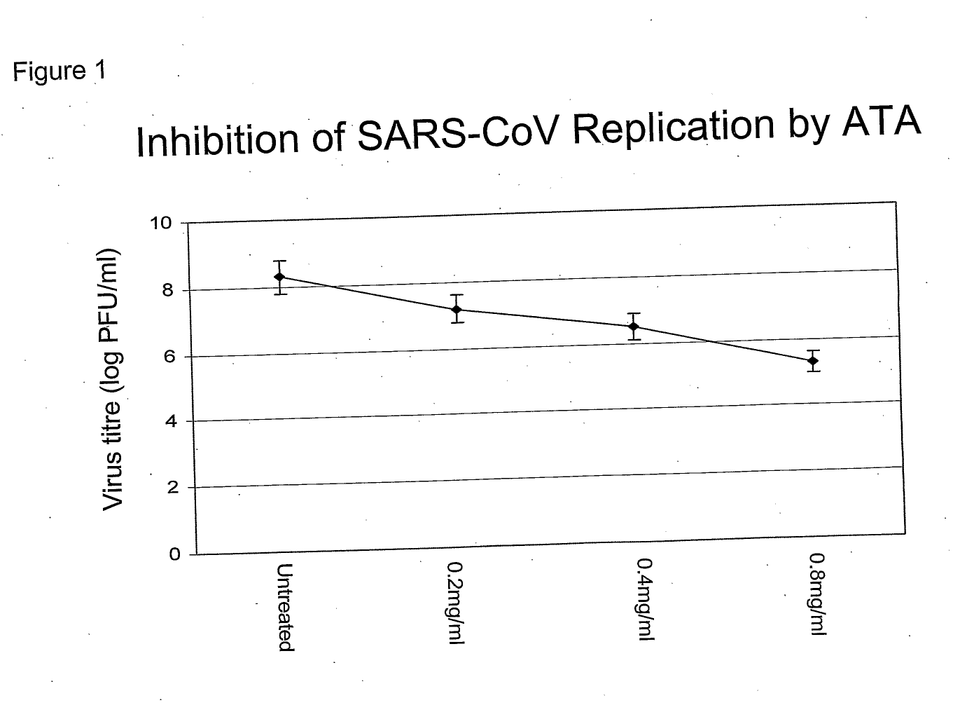

We have tested the anti-SARS-CoV effect of aurintricarboxylic acid (ATA) over a wide range of concentrations, i.e. 0.8 mg/ml, 0.4 mg/ml, 0.2 mg/ml, 0.1 mg/ml and 0.05 mg/ml prepared in Minimum Essential Medium (MEM) with 10 mM HEPES (pH 7.2) and 10% of fetal calf serum. Plaque assays were used to determine the effect of ATA on SARS-CoV replication. Vero cells were infected with SARS-CoV in a 24-well plate; serial dilutions of ATA were added to the infected cells after the initial virus adsorption step. Twenty-four hours post infection, we collected the supernatants from the aforementioned cultures for plaque assays to determine the inhibitory effect of ATA on SARS-CoV replication. As shown in FIG. 1, in comparison with the untreated cells, more than 1,000-fold inhibition of virus replication was observed when the culture was treated with ATA at a concentration of 0.8 mg/ml, while at least 100-fold inhibition was observed at 0.4 mg/ml. An inhibitory effect on viral replication could still be observed at 0.2 mg/ml, with viral replication level being 10 times lower than that of the control.

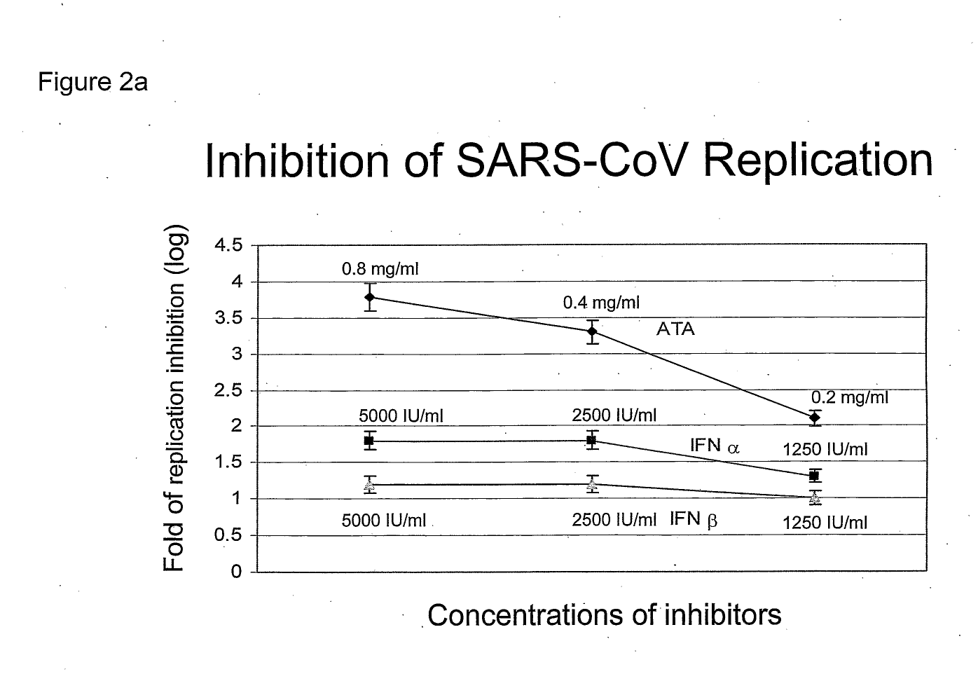

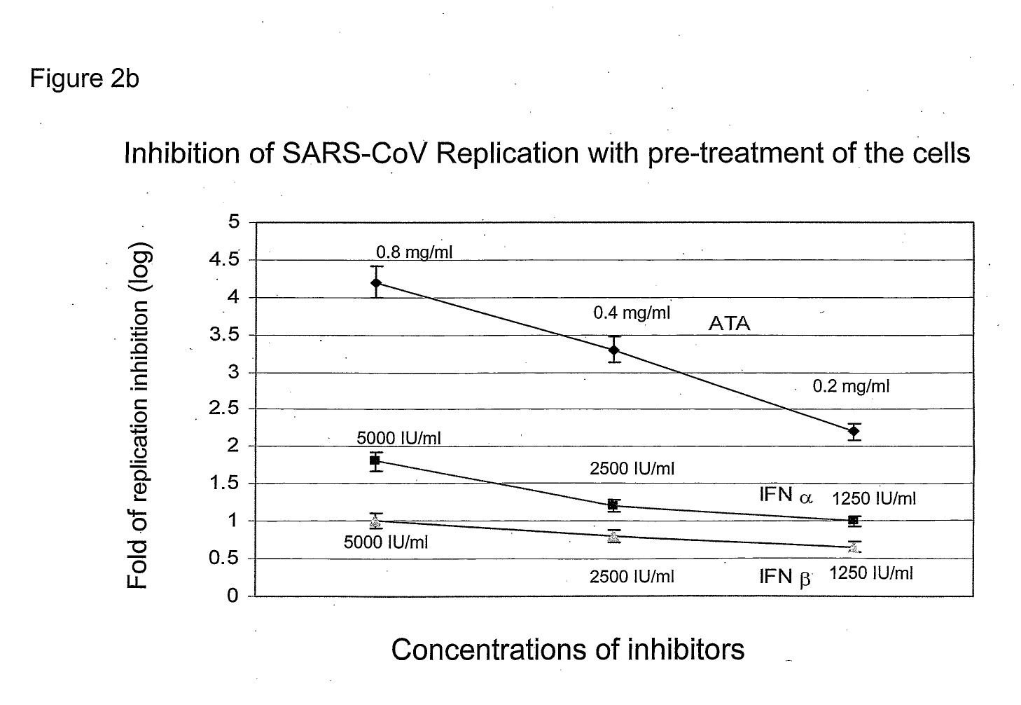

We also compared the inhibitory effect of ATA on SARS-CoV replication with that of IFN α and β. To this end, dilutions of ATA and IFNs at the highest effective concentrations were used to treat the cultures [10,12], followed by determination of virus loads. The virus loads at 24 h post-infection were quantified by real-time RT-PCR analysis using specific primers and probes against SARS-CoV nucleocapsid protein. As shown in FIG. 2a, ATA at 0.8 mg/ml inhibited the virus RNA replication by more than 1,000 fold, versus 100 fold inhibition by interferon α at 5000 IU/ml and 10 fold by interferon β at 5000 IU/ml (FIG. 2a). This result indicates that ATA was about 10 times more potent than interferon α and 100 times more potent that interferon β as an anti-SARS-CoV agent.

To further analyze whether there is a prophylactic effect of ATA, we pre-treated the cells with a series of concentrations of ATA, interferons α and β for 12 h before the adsorption of SARS-CoV to Vero cells and then added the above inhibitors after adsorption. As shown in FIG. 2b, the inhibition effect of ATA and interferons α and β were about the same as samples without the pre-treatment, implicating that the inhibition effect may take place after the virus enter the cells.

### Western Blot Analysis

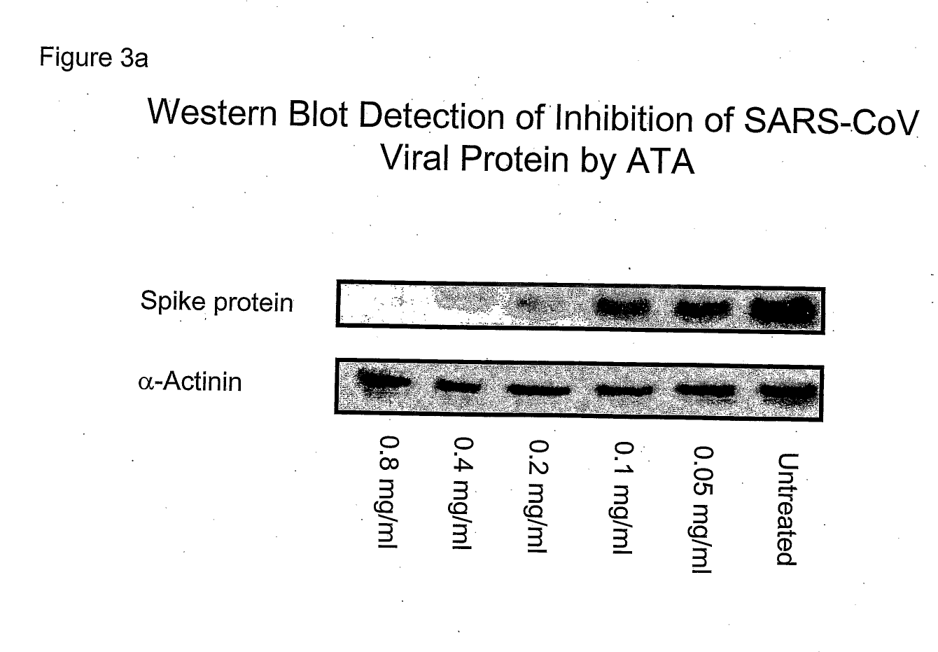

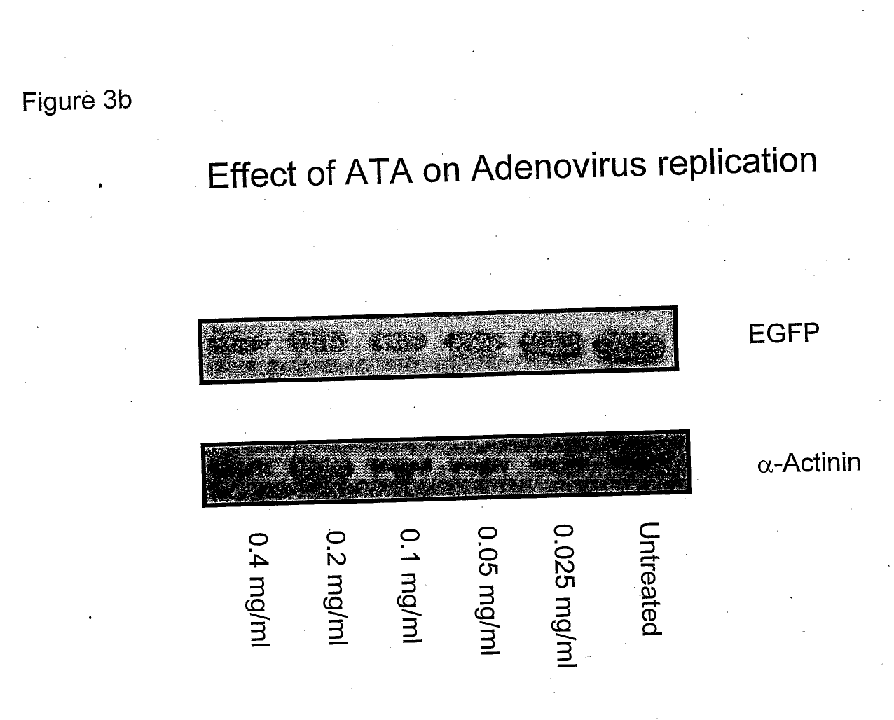

To further confirm the inhibitory effect, we performed Western blot analysis using a monoclonal antibody against SARS-CoV spike (S) protein. As shown in FIG. 3a, the level of the S protein was significantly lower in the ATA treated group than in the untreated group. At a concentration of 0.8 mg/L, ATA blocked viral protein synthesis, confirming that ATA can significantly inhibit viral protein synthesis.

To characterize the specificity of the anti-viral activity of ATA, we also tested the compound for its ability to block protein expression by adenovirus replication. The replication deficient adenovirus type 5 expressing EGFP was used to infect HEK-293 cells. The same concentrations of ATA used in the above-mentioned SARS-CoV inhibition experiments were added to the adenovirus-infected cells. A Western blot analysis was subsequently performed to determine the expression level of EGFP. No significant inhibition was observed in any ATA-treated samples compared with non-ATA-treated cells (FIG. 3b), indicating that the inhibition of SARS-CoV replication by ATA was clearly selective.

### Cell Proliferation Assay

To further explore the therapeutic potentials of ATA, we determined the selectivity index (SI) as defined by the ratio of drug concentration causing cellular toxicity to that producing anti-viral effect. To this end, non-radioisotope cell proliferation analysis system XTT from Roche (Mannheim, Germany) was used. CC₅₀ indicates the concentration that causes 50% of the cytotoxicity, while EC₅₀ means the concentration of inhibitors that inhibited 50% of the virus replication. We found that the SI of ATA is 187 versus 30 of IFN α and 20 of IFN β; indicating ATA is a potent anti-viral compound with low toxicity (table 1).

Because of its low toxicity in cell culture and animals [17, 18], ATA has been evaluated for its anti-viral activities in viruses such as immunodeficiency virus type I [17, 19]. The potency of ATA against SARS-CoV replication appears to be higher that that of the reported chemicals such as glycyrrhizin and recently reported nelfinavir [12, 20]; both drug candidates reported to have two logs or less inhibition effect on SARS-CoV replication, while ATA showed more than three logs of inhibition effect. The biological activities of ATA are believed to be quite complicated, including inhibition of protein synthesis, prevention of the attachment of mRNA to ribosomes in cell-free systems and suppression of enzymes involved in polynucleotide metabolism [21].

As will be apparent to one of skill in the art, vaccine development could take years to complete and serious adverse reactions have been reported in other coronavirus vaccine studies, i.e., exacerbation of disease in animals receiving vaccines prior to infection [22]. Certain precautions have been proposed for the development of SARS-CoV vaccines due to potential detrimental effects [23], meaning that the search for anti-SARS-CoV drugs must be pursued.

The 3D theoretical model for RdRp (RDRP, ID=105S, Xu et al., 2003, NAR 31: 7117-7130), spike protein subunit 1 (S₁, ID=1Q4Z, Spiga et al., 2003, Biochem Biophys Res Commun 310: 78-83), spike protein subunit 2 (S₂, ID=1Q4Y, Spiga et al., 2003) of SARS-CoV, the 3D crystal structure for nucleocapsid protein (N protein, ID=1SSK, Huang et al., 2004, Biochemistry 43: 6059-6063), non-structural protein 9 (Nsp9, ID=1QZ8, Egloff et al., 2004, PNAS 101: 3792-3796), main protease (3Clpro, ID=1Q2W, Anand et al., 2003, Science 300: 1763-1767) of SARS-CoV, the crystal structure for YopH from Yersinia pestis (ID=1QZ0, Sun et al., 2003, J Biol Chem 278: 33392-33399), m-calpain from Rattus norvigecus (ID=1DF0, Strobl et al., 2000, PNAS 97: 588-592), RNA-dependent RNA polymerase from Dsrna Bacteriophage φ6 (ID=1HI8), Rabbit Hemorrhagic Disease Virus (ID=1KHW), Poliovirus (ID=1RDR), Bovine Viral Diarrhea Virus (IVDV, ID=1S4F), Norwalk virus (ID=1SH0) and HIV reverse transcriptase (ID=3HVT) were downloaded from Protein Data Bank. PRODRG2 (Aalten, 2004, Acta Crystallographica D60, in press) and JME editor (http://www.cem.msu.edu/˜reusch/VirtualText/Questions/MOLEDITOR/jme_window.html) were used to derive the atomic coordinates of ATA. VAST and DALI programs were used to locate similar structural patterns between crystal structures of yopH and RdRps. Sequential structural alignment was done by CE (Shindyalov and Bourne, 1998, Protein Eng 11: 739-747) and COMPARER (Sali and Blundell, 1990, J Mol Biol 212: 403-428). Finally, 3D structural comparative analysis was performed by LGA (Zemla, 2003, NAR 31: 3370-3374). Preparation of macromolecule and ligand prior molecular docking was done using WhatiF software (Vriend, 1990, J Mol Graph 8:52-56). Molecular docking to determine the best confirmation in terms of lowest Gibbs free energy and shape complementarity was performed using Autodock 3.0 (Morris et al., 1998, Journal of Computational Chemistry 19: 1639-1662). The visualization of the 3D structural data was generated by Rasmol (Bernstein, TIBS 25: 453-455).

Protein 3D structural alignments were performed on ten amino acid (mainly RdRps) sequences of interest, namely: Hepatitis C virus RNA-dependent RNA polymerase; m-caplain; bacteriophage φ6 RNA-dependent RNA polymerase; rabbit hemorrhagic disease virus RNA-dependent RNA polymerase; SARS-CoV RNA-dependent RNA polymerase; Yersinia pestis phosphatase yopH; poliovirus RNA-dependent RNA polymerase; bovine viral diarrhea virus RNA-dependent RNA polymerase; Norwalk virus RNA-dependent RNA polymerase; and HIV Type 1 RNA-dependent RNA polymerase. Regions with homologous 3D-conformations were identified together with their conserved secondary structures (shown in FIG. 5). In total, there are eight structurally conserved motif blocks (CMBs) with each block extending at least eight amino acid residues. The secondary structures for all CMBs include six α-helices and two β-strands regions. The exact position of all CMBs in each protein are provided in FIG. 5.

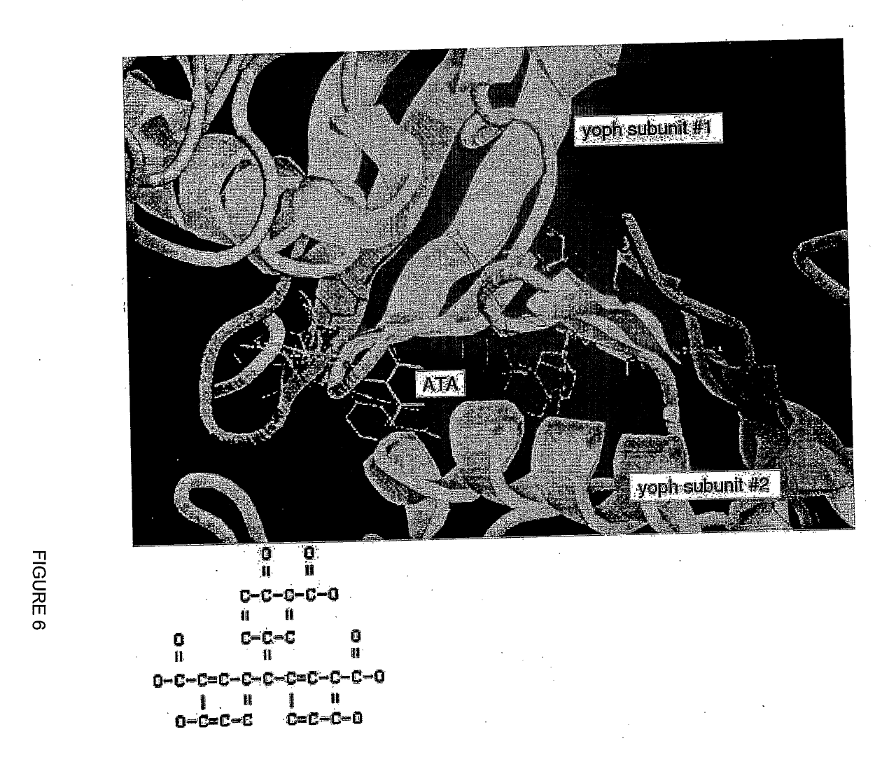

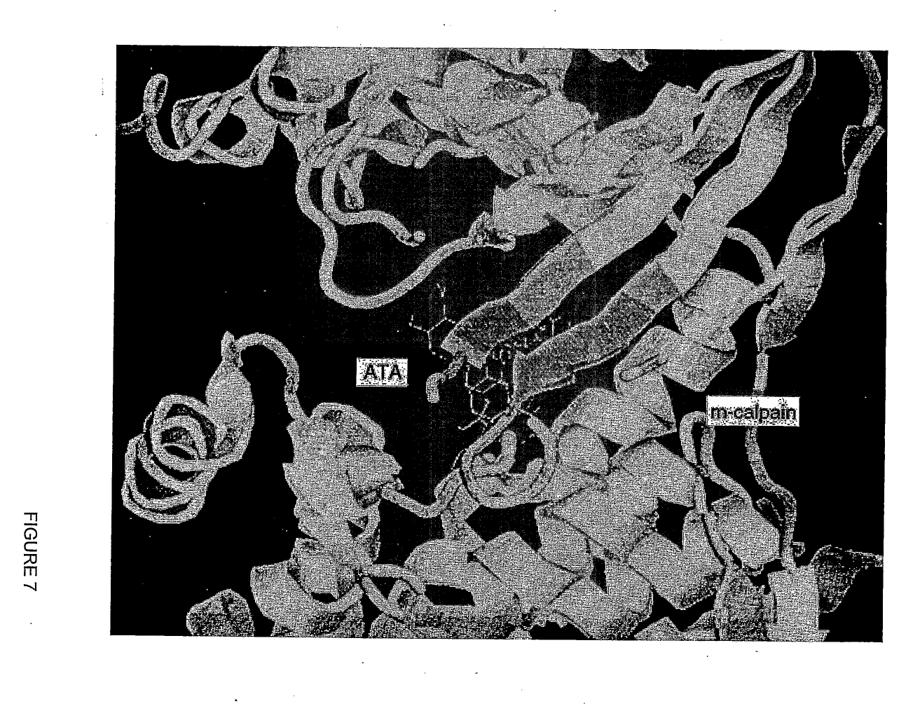

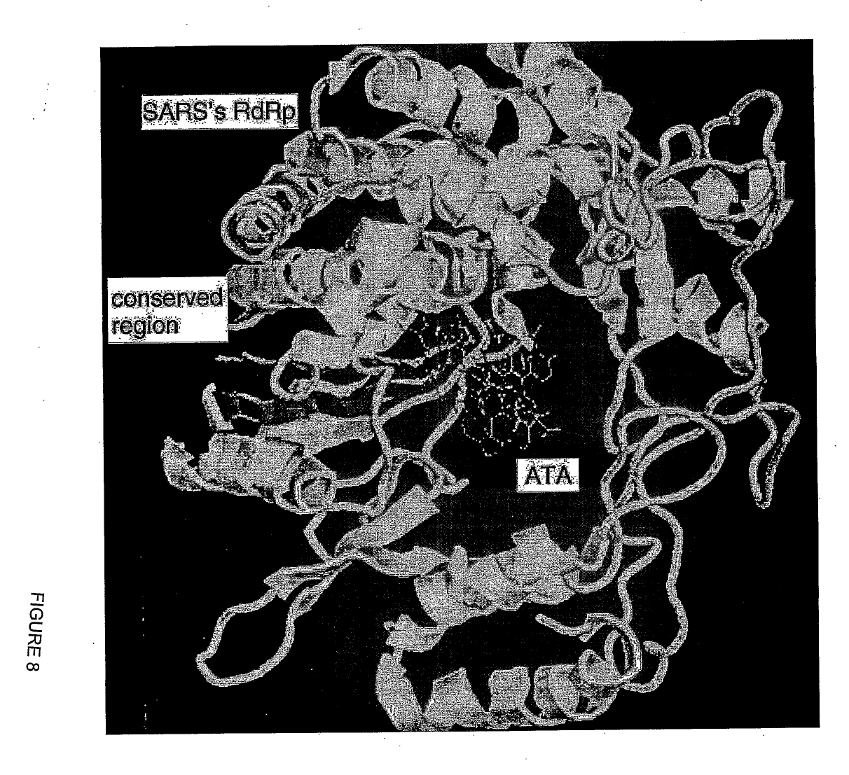

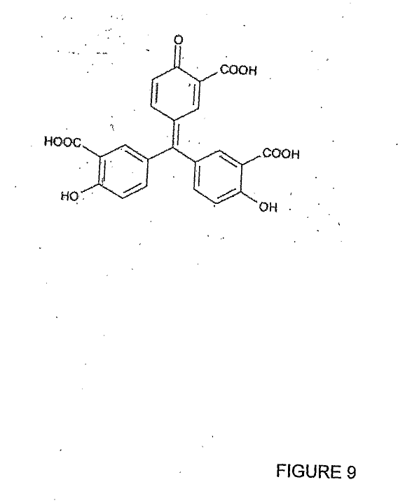

Analysis of the molecular docking method based on free energy of ligand binding, ΔG, between ATA and all proteins (FIGS. 6 and 7) revealed that ATA binds favorably to one structurally conserved region (R_{binding}) among all proteins (FIG. 5). As shown in FIG. 8, the corresponding R_{binding} region in SARS-CoV's polymerase (Ser 754-Tyr 766) overlapped with one CMB. R_{binding} is located in the palm sub-domain and consists mainly of anti-parallel β-strand-turn-β-strand hairpin structures. This conserved region is similar to the majority of the remaining nine proteins in terms of their secondary structures. Surprisingly, this R_{binding} region also contains a highly conserved ‘XSDD’ amino acid motif that is especially prominent among viral RdRps, of which two of the highly conserved aspartic acid (D) residues form the catalytic center important for polymerase activity (Xu et al., 2003).

The free energy of ligand binding (final intermolecular energy+torsional free energy), ΔG, between ATA and all proteins is shown in Table 2. The proteins that were documented to be inhibited by ATA were assigned as positive controls (HIV integrase, yopH and m-calpain), their estimated free energies of binding were −11.88 kcal/mol, −7.79 kcal/mol and −7.67 kcal/mol respectively. Any estimated free energies of binding approximately −7.67 kcal/mol or lower are candidates for inhibition by ATA if the corresponding binding motif includes catalytic sites of that specific protein. When we studied the binding of ATA onto various RNA dependent RNA polymerases from other organisms, the free energy of binding for most RdRps were significantly higher, than −7.67 kcal/mol, and therefore a lower inhibition by ATA (Bovine viral diarrhea virus, Dsrna bacteriophage, Feline calicivirus, Hepatitis C virus, HIV, Poliovirus and Rabbit hemorrhagic disease virus). Only the RdRps from SARS-CoV (ΔG=−7.68) and Norwalk virus (ΔG=−14.92) were estimated to have a lower ΔG (<−7.67 kcal/mol).

It is of note that as discussed above, the R_{binding} domain includes 2 of the 3 predicted RdRp catalytic residues and a highly conserved secondary structure. While not wishing to be bound to a particular theory, the inventors believe that these residues are important for metal ion chelation (Bressanelli et al., 2002, J Virol 76: 3482-3492; Beese and Seitz, 1991, EMBO J 10: 25-33; Huang et al., 1998, Science 282: 1669-1675). Structurally, this binding pocket is located in the palm sub-domain which consists mainly of anti-parallel β-strand-turn-β-strand hairpin structures.

As reported above, there are eight structurally conserved CMBs and their secondary structures include six α-helices and two β-strand regions. Among these structurally conserved regions, we subsequently identified that there was one common region recognized by ATA. The binding of ATA to this region also fulfilled the lowest free energy of ligand binding. The beauty of using this free energy of ligand binding is that we were able to quantify the binding strength between a macromolecule and a ligand. Therefore, if a ligand binds strongly (with lower free energy of ligand binding ΔG) to the active domains of one specific protein, it will presumably inhibit the activity for that specific protein. On the other hand, if ATA does not bind to the active domains of the protein, ATA will not be able to inhibit the function of the protein regardless of the strength of binding.

### One Step Growth Experiment

Hela cells were seeded in a 6 well plate and infected with vaccinia virus (WR) at a multiplicity of infection of 5. After a one hour incubation at 37 C and 5% CO₂, the media was removed and the cell monolayer was washed three times with cold phosphate buffered saline. Next, appropriate dilutions of ATA were made in 1 mL of media and added to the cells. At various time intervals, the cells were removed with a cell scraper and frozen at −80 C. A freeze/thaw method was used to extract the virus from the cells. The supernatant from the freeze/thaw was centrifuged at 2000×g for 5 minutes and the supernatant was collected. Titration of the virus was done on BSC-1 cells. A one-in-ten dilution of the supernatant was made, and 200 μl was applied to the cell monolayer. Cells were fixed after a two day incubation at 37 C, in 5% CO₂. 3%, folmaldehyde was used as a fixative, and the cells were stained with 0.5% crystal violet. The virus titre was calculated by counting the number of plaques, and determining the number of plaque forming units per mL.

### Protein Expression

The vaccinia H1L and A30L open reading frames were amplified by PCR (polymerase chain reaction) from vaccinia virus WR genomic DNA. The H1L N-terminal primer was 5′-TAAAGGATCCATGTACCCATACGATGTTCCAGATTACGCTATGGATAAGAAAA GTTTGTATAAA-3′ (SEQ ID NO. 4). The H1L C-terminal primer was 5′-TTTATACAATAACTATTCTTAATTGAGCTCGCCT-3′ (SEQ ID NO. 5). The A30L N-terminal primer was 5′-AATTGGATCCATGTACCCATACGATGTTCCAGATTACGCTATGGAAGACCTTA ACGAGGCAAACT-3 (SEQ ID NO. 6); and the A30L C-terminal primer was 5′-CCTTCTCTTAAGTTAGCAGCAACTGAGCTCAAAT-3′ (SEQ ID NO. 7). Primers contain a 5′ BamH1 and 3′ Xho1 restriction sites to facilitate cloning. PCR products were ligated into pCR 2.1-TOPO (Invitrogen) and sequenced for fidelity. Coding regions for the genes were then excised with BamH1 and Xho1 and ligated into pre-digested (BamH1/Xho1) pGEX-6P-3. These plasmids were then used to transform E.coli.BL21-pLys-S-DE3. This strain produces lysozyme, which aides in disrupting the cell membrane for protein purification. 500 mL cultures were inoculated with an overnight culture and allowed to grow at 37 C until an OD of 0.7 was reached at 600 nm. Isopropyl-B-D-thiogalactopyranoside (IPTG) was then added to a final concentration of 1 mM and expression of GST-A30L and GST-H1L fusion proteins was continued for 3 hours. For reasons unknown, a fresh transformation of the H1L-pGEX construct into the cells had to be carried out for each new purification. Plating out a glycerol stock of the recombinant bacteria, and performing protein expression from here resulted in low quantities of protein.

### Protein Purification

Induced cultures expressing GST-A30L and GST-H1L were pelleted by ultra-centrifugation at 5000×g for 15 minutes. Pellets were then lysed in 50 mM Tris-HCl, pH 8.0, 150 mM NaCl, 2 mM EDTA and 1 mM DTT. Mini-Tab (Roche) protease inhibitor tablets were also added. Extra lysozyme was added to 5 mM in order to facilitate cell lysis. The solution was then sonicated, to shear the DNA present, which can clog purification columns. The soluble protein fraction was isolated by ultra-centrifugation at 18000 rpm for 25 minutes, and filtered through a 0.45 μM syringe filter (Corning). Fractions were then loaded onto an AKTA UPC-900 fast performance liquid chromatography (FPLC) unit (Amersham). Protein samples were run through a GSTrap FF purification column (Amersham) at a flow rate of 0.5 mL/min. The column was previously washed with 0.01M Phosphate buffered saline (PBS), pH 7.2. The GST fusion proteins were eluted in 1 mL fractions with 50 mM Tris-HCl, 10 mM reduced glutathione, pH 8.0. The purity of these preparations was assessed by SDS-polyacylamide gel electrophoresis and determined to be approximately 90%. The fraction containing the protein of interest was then further purified with QFF anion exchange columns (Amersham). The protein was loaded in the GST column elution buffer, and eluted in 50 mM Tris-HCl via a salt gradient. Samples of the eluted fraction were then run on a gel and the fractions containing the protein of interest were stored. Purity was estimated to be greater that 95%.

### RT-PCR Analysis

mRNA levels were analyzed using an Applied Biosystems 7300/7500 Real Time PCR System, following the manufacturer's protocol. Hela cells were grown in the presence or absence of ATA for 3 hours, and supernatant samples were collected at 0, 2, 6, 12 hrs. RNA was purified with an Rneasy Kit (Qiagen). 1 μg of viral RNA was used to detect F10L mRNA levels. As a control, the levels of GADPH were also followed.

### H1L Kinetic Analysis

Before characterizing the inhibition of H1L by ATA, it was necessary to determine the Km and Vmax values of the enzyme. These kinetic parameters are then used to optimize conditions for an IC50 inhibition assay. To obtain these values, enzyme activity assays were performed in 96 well plates. The assays were performed in 25 mM HEPES, 50 mM NaCl, 5 mM DTT, 2.5 mM EDTA in a final volume of 200 μL. The first step involved the addition of buffer, and 1.5 μg of enzyme. The Bradford Method was used to quantify the purified protein. Additionally, 25 μg of BSA was added to stabilize the enzyme and create optimal kinetic conditions. Reactions were pre-incubated at 37 C for 15 minutes in a water bath. Substrate was then added to start the reaction. Obtaining the Vmax and Km values requires the plotting of a Michealis-Menton curve, in which then enzyme concentration is held constant while the substrate concentration varies. Substrate concentrations used were 0.06, 0.125, 0.25, 0.5, 1, 2 and 4 mM pNPP. These were the concentration reached in the final 200 μl reaction. A further incubation of ten minutes followed addition of substrate. The reaction was stopped by the addition of 13% K₂HPO₄. Absorbances were read with a SpectraMax Plus (Molecular Devices), at 405 nm. The Km and Vmax values were calculated from the Lineweaver-Burk curve of the data. The substrate, pNPP (p-nitrophenylphosphate), absorbs at 405 nm, so a set of control reactions was also run. These involved addition of substrate but no enzyme in the first step. The values obtained in the controls were subtracted from the appropriate samples.

### IC50 Determination

Assays were performed in the buffer previously mentioned. Reactions were carried out at a fixed substrate concentration, equal to the Km value of the enzyme. ATA was used at varying concentrations, ranging from 2 to 125 μM. The reaction sequence is the same as described above. Since ATA and pNPP absorb at 405 nm, a series of control reactions, representing the dilutions of ATA and pNPP used were set up. These reactions did not receive enzyme, and the absorbance value read from them was then subtracted from the appropriate samples. The absorbance at 405 nm was plotted against the concentration of ATA used. The IC50 value is that concentration at which half-maximal enzyme activity occurs.

### ATA Inhibits Replication of Vaccinia Virus in Tissue Culture

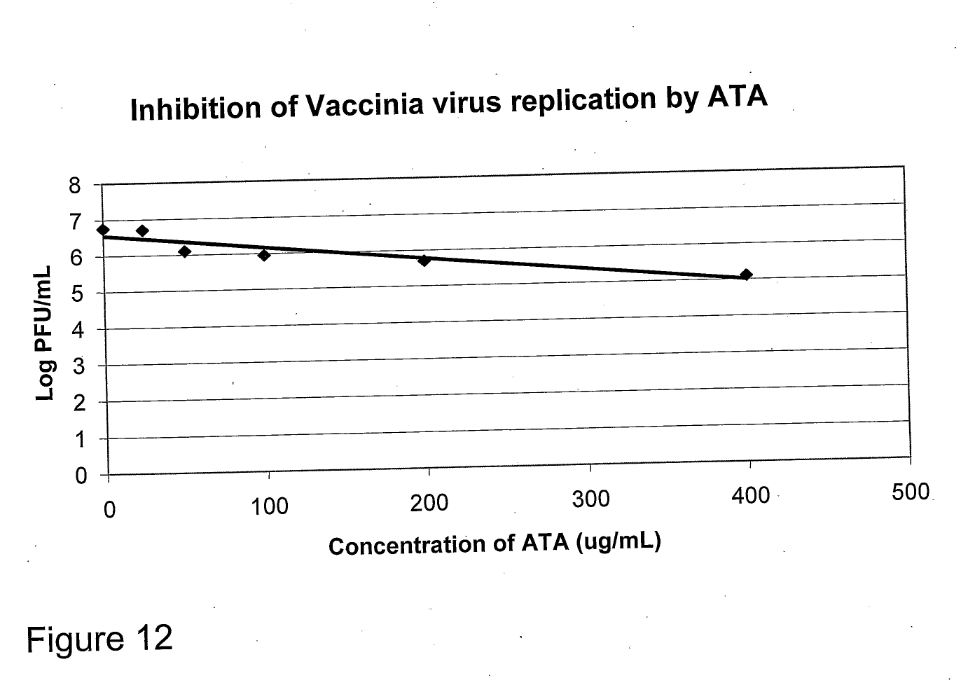

The effect of ATA on vv replication was studied in numerous cell lines using plaque assays. A range of concentrations from 0 to 400 μg/mL was used. Hela cells were infected with vv (WR) at a MOI of 10 (FIG. 12). The viral load dropped by over one log value when 400 μg/mL of ATA was used compared to the control with no drug. Replication inhibition was seen in the lowest dilutions used. Infectious virus counts steadily decreased as the concentration of the drug was increased. Huh7, Vero and Rk13 cell lines were also used to repeat the same experiments. In each case, the results were similar. All experiments were performed in triplicate.

To further characterize this inhibition, a time-course inhibition assay was performed with RK13 cells. The cells were infected with vv (WR) at a MOI of 5. ATA was applied at various concentrations, and the PFU/mL was determined at 0, 4, 24, 48, 72 and 96 hours post-infection (FIG. 13). In the absence of ATA, the levels of virus increased to a maximum of 1.55×10 pfu/mL at 48 h.p.i., and then subsequently decreased. When ATA was used at 400 ug/mL, the viral load again peaked at 48 h.p.i., with the plaque count being 616 pfu/mL. This represents a 250,000 fold difference in treated versus untreated cells. At the final time point (96 hrs.), the viral count in the treated sample was only 0.00026% of the viral count in the untreated control.

### ATA Inhibits the H1L Phosphatase

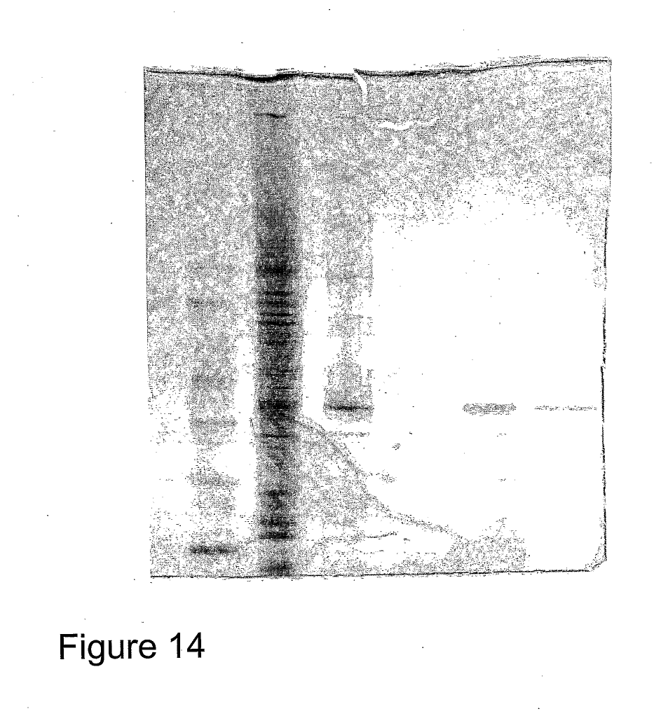

ATA has previously been shown to inhibit phosphatases, including those from both eukaryotic and prokaryotic sources. To further characterize the mechanism behind the replication inhibition, it was decided to perform enzyme inhibition assays. These assays specifically detect phosphatase activity towards tyrosine residues. The VV H1L phosphatase was purified to near homogeneity (FIG. 14) as described in materials and methods.

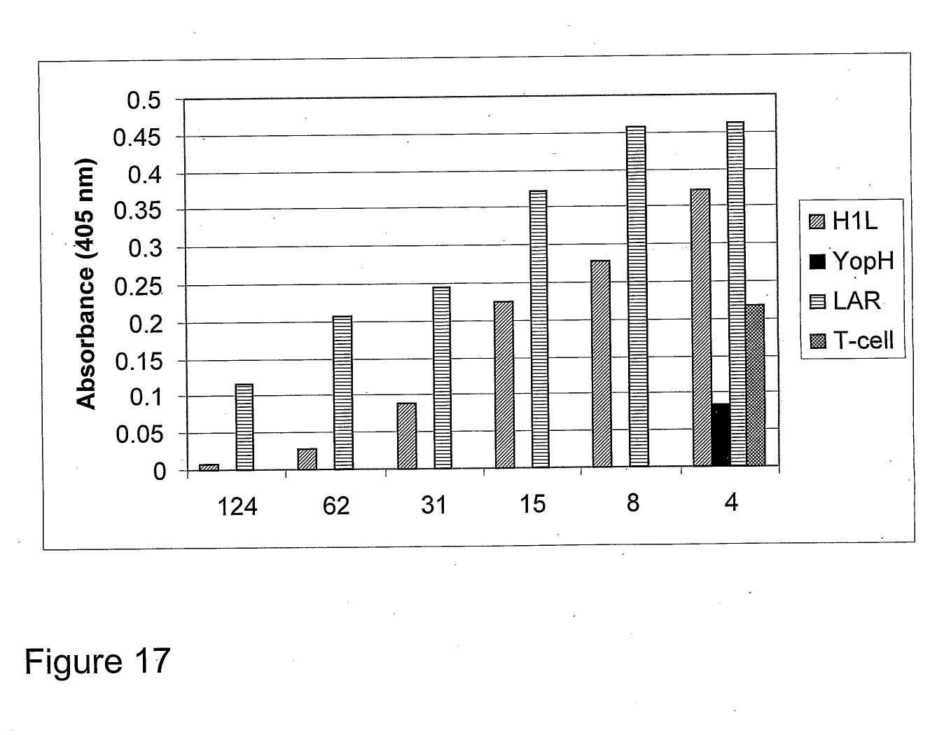

All assays were performed using the GST-H1L fusion protein. Previous studies have shown that the fusion protein and H1L alone have nearly identical activities (37). As a control, preparations of GST-A30L, were also run in identical phosphatase assays. These reactions always gave results comparable to the blank. Assays were first performed to determine the Vmax and Km of H1L towards the experimental substrate, para-nitrophenylphosphate. The product released, para-nitrophenolate, results in the yellow colour of the reaction (33), which is quantified by following the absorbance at 405 nM. The resulting data collected was fit to a Lineweaver-Burk plot, with the Vmax being 6.40 (abs. units) and a Km of 1.00 mM pNPP (FIG. 15). These values are solved from the formula provided in FIG. 17, with the Vmax representing the y-axis value and the Km represented by the x-axis value.

To determine the extent to which ATA inhibits the activity of H1L, IC50 assays were performed. The IC50 value was found to be 16 μM (FIG. 16). The IC50 value of ATA against other PTPs (protein tyrosine phosphatases) has been previously described (32, 46).

The potency of ATA towards H1L and other phosphatases was also compared. The enzyme activity of H1L, LAR, YopH and T-cell phosphatases was standardized, such that for the assay, the same amount of enzyme activity was added to each reaction. It was found that ATA was more selective for the YopH and T-cell phosphatase and less so for LAR, when compared to H1L (FIG. 17). Activity of the YopH and T-cell phosphatases only became detectable at the lowest concentration of ATA used, 4 μM, while H1L and LAR still showed activity at the highest concentration of 124 μM.

While the preferred embodiments of the invention have been described above, it will be recognized and understood that various modifications may be made therein, and the appended claims are intended to cover all such modifications which may fall within the spirit and scope of the invention.

### REFERENCES

[1] Marra et al., 2003, Science. 300 (5624), 1399-1404.
[2] Rota et al., 2003. Science. 300 (5624), 1394-1399.
[3] Nicholls et al., 2003, Respirology. Suppl:S6-8.
[4] Holmes K V,. 2001 Coronaviruses. In: Knipe D M and Howley P M (Eds), Field's Virology. Lippincott, Williams & Wilkins, Philadelphia, Vol. I, pp. 1187-1203.
[5] Hierholzer, 1976, Virology. 75(1):255-65.
[6] Bucknall et al., 1972, Proc Soc Exp Biol Med. 139(3):811-7.
[7] Van Der Hoek et al., 2004, Nat Med. 10(4):368-73.
[8] He et al., 2003, Biochem Biophys Res Commun. 28;311(4):870-6.
[9] He et al., 2004, Biochem Biophys Res Commun 316(2) 476-483
[10] Tan et al., 2004, Emerg Infect Dis. 10(3) 581-586.
[11] Stroher et al., 2004, J Infect Dis. 189(7):1164-7.
[12] Cinatl et al., 2003, Lancet. 14;361(9374):2045-6.
[13] Talib and Hearst, 1983, Nucleic Acids Res. 11(20): 7031-42.
[14] Siegelman and Apirion, 1971, J Bacteriol. 105(3):902-7.
[15] Stewart et al., 1971, Proc Natl Acad Sci USA. 68(1):97-101.
[16] Schmidt et al., 1979, J Clin Microbiol. June 1979; 9(6):722-8.
[17] Balzarini et al., 1986, Biochem Biophys Res Commun. 14;136(1):64-71.
[18] Roberts-Lewis et al., 1993, J Neurochem. 61(1):378-81.
[19] Cushman and Sherman, 1982, Biochem Biophys Res Commun. 29;185(1):85-90.
[20] Yamamoto et al., 2004, Biochem Biophys Res Commun. 318(3):719-25.
[21] Mellon, 1984, Biochem Pharmacol. 33(7):1047-57.
[22] Cavanagh, 2003, Avian Pathol 32(6):567-82.
[23] Marshall and Enserink, 2004, Science. 303(5660):944-6.
[24] Alonso et al., 2001, J. Bio. Chem. 276, 4766-4771.
[25] Becker et al., 1972, Anti. Mic. and Chemo. 1, 483-488.
[26] Benchokroun et al., 1995, Biochem. Pharm. 49, 305-313.
[27] Broyles, 2003, J. Gen. Virol. 84, 2293-2303.
[28] Broyles et al., 2004, J. Virol. 78, 2137-2141.
[29] Byrd et al., 2004, J. Virol. 78, 12147-12156.
[30] Clercq and Neyts, 2004, Rev. Med. Virol. 14, 289-300.
[31] Clercq, 2001, Clin. Mic. Rev. 14, 382-397.
[32] Cho et al., 2004, Mol. Cells. 18, 46-52.
[33] Denu et al., 1995, J Biochem. 270, 3796-3803.
[34] Denu and Dixon, 1995, Proc. Natl Acad. Sci. 92, 5910-5914.
[35] Derrien et al., 1999, J. Virolgy. 73, 7287-7296.
[36] Diperna et al., 2004, J. Bio. Chem. 279, 36570-36578
[37] Guan et al., 1991, Nature. 350, 359-362.
[38] Hakes et al., 1993, Proc. Natl. Acad. Sci. 90 4017-4021.
[39] He et al., 2003, Biochem. Biophy. Res. Comm. 311, 870-876.
[40] He et al., 2004, Biochem. Biophys. Res. Comm. 320, 1199-1203.
[41] Huang et al., 2002, EMBO rep. 3, 1088-1093.
[42] Hunt and Wagner, 1975, J. Virol. 16, 1146-1153.
[43] Karp, 2002, Cell and molecular biology. John Willey, New York.
[44] Kern, 2003, Antiviral Reas. 57, 35-40.
[45] Lehninger A., Nelson D., Cox. (1993). Principles of Biochemistry. Worth publishers, New York, N.Y.
[46] Liang et al., 2003, J. Biochem. 278, 41734-41741.
[47] Liu et al., 1995, J. Virol. 69, 7823-7834.
[48] McCune et al., 1989, Biochem. J. 259, 925-927.
[49] Mercer and Traktman, 2003, J. Virol. 77, 8857-8871.
[50] Moss, 2001, Poxviridae: The viruses and their replication. Fields Virology. 2849-2855.
[51] Mossman et al., 1995, Virol. 206, 572-582.
[52] Najarro et al., 2001, J. Virol. 75, 3185-3196.
[53] Rui et al., 1998, J. Biochem. 273, 28-32.
[54] Shisler and Jin, 2004, J. Virol. 78, 3553-3560.
[55] Tian et al., 1998, Science. 281, 257-259.
[56] Todd et al., 1999, J. Bio. Chem. 274, 13271-13280.
[57] Wang et al., 2004, Nature Imm. 1-9

TABLE 1
Selection of Inhibitors

| | CC50 | | EC50 | | SI |
|---|---|---|---|---|---|
| ATA | 37.5 | mg/ml | 0.2 | mg/ml | 187 |
| IFNα | 15000 | IU/ml | 500 | IU/ml | 30 |
| IFNβ | 10000 | IU/ml | 500 | IU/ml | 20 |

TABLE 2
Estimated Free energy
(final intermolecular energy + torsional free energy)
of different targeted proteins in complex with ATA using Autodock.

| | | | Estimated |
|---|---|---|---|
| | | | Free Energy |
| | | | of Binding |
| | | | in Complex |
| | | Type of | with ATA/ |
| | Targeted Protein | structure used | kcal/mol |
| RNA | RdRp (Bovine Viral | Crystal Structure | −5.82 |
| dependent | Diarrhea Virus) | | |
| RNA | RdRp (Dsrna Bacterio- | Crystal Structure | −5.87 |
| polymerase | phage) | | |
| | RdRp (Feline calicivirus) | Theoretical model | −5.83 |
| | RdRp (Hepatitis C Virus) | Crystal Structure | −5.8 |
| | RdRp (HIV) | Crystal Structure | −5.82 |
| | RdRp (Norwalk virus) | Crystal Structure | −14.92 |
| | RdRp (Poliovirus) | Crystal Structure | −5.83 |
| | RdRp (Rabbit Hemor- | Crystal Structure | −5.94 |
| | rhagic Disease Virus) | | |
| | RdRp (SARS-CoV) | Theoretical model | −7.68 |
| Protein | Yoph* | Crystal Structure | −7.79 |
| known to | HIV integrase* | Crystal Structure | −11.88 |
| be inhibited | m-Calpain* | Crystal Structure | −7.67 |
| by ATA* | | | |
| Other | Main Protease (3CL) | Crystal Structure | −7.78 |
| SARS' | N Protein | Crystal Structure | −8.32 |
| proteins | Nsp 9 | Crystal Structure | −7.64 |
| | S₁ [28] | Theoretical model | −18.59 |
| | S₂ [28] | Theoretical model | −7.66 |
| | S₁ (unpublished model) | Theoretical model | −14.79 |
| | S₂ (unpublished model) | Theoretical model | −15.22 |

1. A method of preparing a medicament for treating an individual infected with or suspected of being infected with an ATA-sensitive virus comprising mixing an effective amount of aurintricarboxylic acid (ATA) or a derivative thereof with a suitable excipient.

2. The method according to claim 1 wherein the ATA-sensitive virus is selected from the group consisting of a coronavirus, a poxvirus, West Nile virus, Norwalk virus, Dengue virus and Japanese Encephalitis virus.

3. A method of treating an individual infected with or suspected of being infected with an ATA-sensitive virus comprising administering to an individual in need of such treatment an effective amount of aurintricarboxylic acid (ATA) or a derivative thereof.

4. The method according to claim 3 wherein the ATA-sensitive virus is selected from the group consisting of a coronavirus, a poxvirus, West Nile virus, Norwalk virus, Dengue virus and Japanese Encephalitis virus.

5. The method according to claim 4 wherein the coronavirus is SARS-CoV.

6. A method of identifying an organism inhibited by ATA comprising:
searching a protein structure database for a peptide of interest having a region homologous to R_{binding} region of SARS-CoV RdRp; and
determining if the homologous region contains catalytic residues for the protein of interest.

7. A method of screening organisms of interest for inhibition by ATA comprising:
incubating an organism of interest under appropriate growth conditions in the presence of ATA; and
determining if growth of the organism of interest has been inhibited.

8. A method of inhibiting growth of an organism comprising administering an effective amount of ATA or a derivative thereof, wherein the organism comprises an essential protein having a region homologous to R_{binding} region of SARS CoV RdRp, wherein said homologous region comprises at least one catalytic residue of the protein.


SEQUENCE LISTING (7 sequences)

SEQ ID NO: 1 - DNA, 16 bp, Artificial
  PCR primer
accccaaggtttaccc

SEQ ID NO: 2 - DNA, 21 bp, Artificial
  PCR primer
accagaatggaggacgcaatg

SEQ ID NO: 3 - DNA, 25 bp, Artificial
  PCR primer
gctgtgaaccaagacgcagtattat

SEQ ID NO: 4 - DNA, 64 bp, Artificial
  PCR primer
taaaggatccatgtacccatacgatgttccagattacgctatggataagaaaagtttgtataaa

SEQ ID NO: 5 - DNA, 34 bp, Artificial
  PCR primer
tttatacaataactattcttaattgagctcgcct

SEQ ID NO: 6 - DNA, 65 bp, Artificial
  PCR primer
aattggatccatgtacccatacgatgttccagattacgctatggaagaccttaacgaggcaaact

SEQ ID NO: 7 - DNA, 34 bp, Artificial
  PCR primer
ccttctcttaagttagcagcaactgagctcaaat